(19) United States
(12) Patent Application Publication
(10) Pub. No.: US 2017/0128489 A1
(43) Pub. Date: May 11, 2017

(54) ORGANO-TRANSITION METAL COMPLEXES FOR THE TREATMENT OF VIRAL INFECTIONS

(71) Applicants: David D. BUSATH, Orem, UT (US); Nathan A. GORDON, Aurora, CA (US); Roger G. HARRISON, Orem, UT (US); Kelly McGUIRE, Provo, UT (US); James H. CLARK, Bountiful, UT (US); Spencer K. WALLENTINE, Provo, UT (US)

(72) Inventors: David D. BUSATH, Orem, UT (US); Nathan A. GORDON, Aurora, CA (US); Roger G. HARRISON, Orem, UT (US); Kelly McGUIRE, Provo, UT (US); James H. CLARK, Bountiful, UT (US); Spencer K. WALLENTINE, Provo, UT (US)

(73) Assignee: Brigham Young University, Provo, UT (US)

(21) Appl. No.: 15/318,198

(22) PCT Filed: Jun. 12, 2015
(86) PCT No.: PCT/US15/35604

§ 371 Date: Dec. 12, 2016

Related U.S. Application Data
(60) Provisional application No. 61/997,888, filed on Jun. 12, 2014.

(51) Int. Cl. A61K 33/34; C07F 3/06; A61K 33/24; C07F 15/04; A61K 33/30; C07F 1/08; C07F 15/06

(52) U.S. Cl. CPC A61K 33/34; A61K 47/48076; C07F 1/08; C07F 3/06; C07F 15/065; C07F 15/045; A61K 33/30; A61K 33/24

(57) ABSTRACT

Organo-transition metal complexes possess anti-viral inhibitory activity against influenza A, including the S3 IN mutant. The organo-transition metal complexes include a transition metal and at least one ligand based on the structure of an M2 proton channel blocker. Compounds and pharmaceutical compositions are useful for treating viruses such as influenza A.

### BACKGROUND

1. Technical Field

The present invention relates to organo-transition metal complexes and compositions thereof, and their use in the treatment of viral infections such as influenza.

2. Background Information

Influenza A causes thousands of deaths annually due to viral infection-related complications. The antiviral amantadine (AMT) functions by blocking proton transport through the M2 channel in influenza A. However, recently drug resistance has developed for AMT due to a serine-to-asparagine mutation at position 31 in M2. The resistance of the virus correlates with reduced block of proton currents in voltage-clamped cells transfected with S31N M2 and reversion solely at M2 position 31 restores efficacy against the stubborn A/WSN/33 strain of influenza for AMT and several AMT analogs. After much effort, two 2,4-disubstituted adamant-1-yl-benzyl-amine compounds were found that exhibit reasonable efficacy against full length M2 WT and M2 S31N in both voltage-clamped Xenopus oocytes and viral plaque reduction assays, but blocking the M2 target reliably continues to be an important scientific and therapeutic challenge.

M2 has recently been structurally investigated as a target for metal ion drug candidates. Among various metal ions that were tested, copper caused the best M2 inhibition. Monovalent copper ions administered at 50 μM reduced M2 activity by 71% while divalent copper ions administered at 500 μM reduced M2 activity by 95%. Both ions reduced M2 activity by binding to the His37 tetrad located within the homotetramer, which confers proton-selectivity to ion transport by the M2 channel in conjunction with the Trp41 tetrad. Residues His37 and Trp41 are completely conserved among strains of influenza A. Compared to AMT, however, free copper ions exhibit high toxicity at concentrations of therapeutic interest. Cu⁺ is unstable in the oxidizing environment of the respiratory tract, and would readily be oxidized to Cu²⁺ in vivo.

In view of the development of drug resistance to M2 channel blockers like AMT, and the toxicity limitations associated with metals ions, there is therefore a need for improved agents to treat influenza with reduced toxicity and reduced susceptibility to drug resistance.

### SUMMARY

The present invention relates to organo-transition metal complexes, and compositions thereof, for use in treating influenza virus. Provided are compounds of formula (I), or salt thereof, and pharmaceutical compositions comprising a pharmaceutically acceptable carrier and a compound of formula (I), or salt thereof

(L¹)ₘM^{P+}(L²)ₙ (I)

wherein:

M is a transition metal, where p is an integer from 0 to 5;

each L¹ is independently a derivative of an M2 proton channel blocker capable of complexing with M, L¹ being a bidentate or tridentate ligand;

each L² is independently an auxiliary ligand, L² being a monodentate, bidentate, tridentate, or tetradentate ligand;
m is 1, 2, or 3; and

n is an integer from 0 to 4.

In a first aspect of the invention are provided pharmaceutical compositions comprising a pharmaceutically acceptable carrier and a compound of formula (I), or salt thereof

(L¹)ₘM^{p+}(L²)ₙ (I)

wherein

M is a transition metal, where p is an integer of from 0 to 5;
m is 1, 2, or 3;

each L¹ is independently a) G¹-Y²—N(R¹)—Y¹—X¹; b) G¹-Y²—N(—Y¹—X¹)₂; or c) G²(-Y¹—X¹)ᵣ, wherein r is 1 or 2;

each R¹ is independently H or C₁₋₆alkyl;

each X¹ is independently OH, OC₁₋₄alkyl, NH₂, NH(C₁₋₄alkyl), N(C₁₋₄alkyl)(C₁₋₄alkyl), COOH, CONH₂, CONH(C₁₋₄alkyl), CON(C₁₋₄alkyl)(C₁₋₄alkyl), C(NH)NH₂, NHC(NH)NH₂, NHOH, SH, S(C₁₋₄alkyl), C(NC₁₋₄alkyl), a 5- or 6-membered nitrogen-containing heteroaryl, or a 4- to 8-membered nitrogen-containing heterocycle, or salts thereof, the 5- or 6-membered nitrogen-containing heteroaryl and the 4- to 8-membered nitrogen-containing heterocycle each being independently optionally substituted with 1-4 substituents independently selected from the group consisting of C₁₋₄alkyl, C₁₋₄haloalkyl, halo, C₁₋₄alkoxy, and C₁₋₄haloalkoxy;

each Y¹ is independently a C₁₋₃alkylene or a bond;

each Y² is independently a bond or C₁₋₃alkylene, the C₁₋₃alkylene being optionally substituted with hydroxy, NH₂, NH(C₁₋₄alkyl), or N(C₁₋₄alkyl)(C₁₋₄alkyl);

G¹ is

a) an alicyclyl, the alicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;

b) a heteroalicyclyl, the heteroalicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;

c) a silacyclyl, the silacyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;

d) C₆₋₂₀aryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy; or

e) a 5- to 20-membered heteroaryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;

G² is

a) a heteroalicyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy; or

b) a silacyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;

each L² is independently an auxiliary ligand, L² being a monodentate, bidentate, tridentate, or tetradentate ligand; and

n is an integer from 0 to 4.

In a second aspect of the invention is provided a compound of formula (I), as described above with the proviso that the compound of formula (I) excludes:

diaqua(N-(1-adamantyl)-iminodiacetate)copper(II);

bis-[(imidazole)(N-(1-adamantyl)-iminodiacetate)copper(II)];

(2,2′-bipyridine)(N-(1-adamantyl)-iminodiacetate)copper(II);

((1S,2S,3S,5R)-2,6,6-trimethyl-N-((1-methyl-1H-imidazol-2-yl)methyl)-bicyclo[3.1.1]heptan-3-amine)copper(II)diacetate or hydrate or solvate thereof;

and

((1S,2S,3 S,5R)-2,6,6-trimethyl-N-((1-methyl-1H-imidazol-2-yl)methyl)-bicyclo[3.1.1]heptan-3-amine)copper(II)dichloride or hydrate or solvate thereof.

In a third aspect of the invention are provided methods of treating influenza A by administration of a composition or compound according to the first or second aspects to a patient in need thereof.

In a fourth aspect of the invention are provided methods of inhibiting the M2 proton channel comprising contacting a cell containing an M2 proton channel with a composition or compound according to the first or second aspects of the invention.

### DETAILED DESCRIPTION

### Definition of Terms

The term “alkyl” as used herein, means a straight or branched chain saturated hydrocarbon. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, means a divalent group derived from a straight or branched chain hydrocarbon. Representative examples of alkylene include, but are not limited to, —CH₂—, —CH₂CH₂—, —CH₂CH₂CH₂—, —CH₂CH(CH₃)CH₂—, and —CH₂CH(CH₃)CH(CH₃)CH₂—.

The term “alkoxy” as used herein, means an alkyl group, as defined herein, appended to the parent molecular moiety through an oxygen atom. Representative examples of alkoxy include, but are not limited to, methoxy, ethoxy, propoxy, isopropoxy, butoxy, isobutoxy, tert-butoxy, pentyloxy, and hexyloxy.

The term “haloalkyl,” as used herein, means, an alkyl group, as defined herein, in which one, two, three, four, five, six, or seven hydrogen atoms are replaced by halogen. For example, representative examples of haloalkyl include, but are not limited to, 2-fluoroethyl, 2,2-difluoroethyl, trifluoromethyl, 2,2,2-trifluoroethyl, 2,2,2-trifluoro-1,1-dimethylethyl, and the like.

The term “aryl,” as used herein, means an all-carbon ring system containing at least one aromatic ring (e.g., phenyl, naphthyl, dihydronaphthalenyl, tetrahydronaphthalenyl, indanyl, indenyl, anthracenyl, phenanthrenyl, 9-methyl-5,6,8,9,10,11-hexahydro-7H-5,9:7,11-dimethanobenzo[9]annulen-7-yl). In some embodiments, the aryl is a C₆₋₂₀aryl. In other embodiments, the aryl is a C₆₋₁₄aryl. In other embodiments, the aryl is a C₆₋₁₂aryl. In other embodiments, the aryl is phenyl or napthyl. The aryl is attached to the parent molecular moiety through any carbon atom contained within the aryl.

The term “alicyclyl” or “alicycle,” as used herein, means an aliphatic cyclic hydrocarbon, i.e., an aliphatic carbocycle. The alicyclyl is non-aromatic but may have one or more carbon-carbon double bonds depending on the particular ring system. Alicyclyl includes, for example, a monocyclic cycloalkyl, a monocyclic cycloalkenyl, a bicyclic cycloalkyl, a bicyclic cycloalkenyl, a tricyclic cycloalkyl, or higher polycyclic cycloalkyls (e.g., tetracyclic, pentacyclic, etc.), each of which may be joined to a second alicyclic ring to form a spirocyclic ring system (i.e., a spirocyclic cycloalkyl). In some embodiments, the alicyclyl has from three to thirty-two carbon ring atoms. In other embodiments, the alicyclyl has from three to sixteen carbon ring atoms, i.e., C₃₋₁₆alicyclyl. In other embodiments, the alicyclyl has from three to twelve carbon ring atoms, i.e., C₃₋₁₂alicyclyl. In other embodiments, the alicyclyl has from three to ten carbon ring atoms, i.e., C₃₋₁₀alicyclyl. In other embodiments, the alicyclyl has from six to twelve carbon ring atoms (C₆₋₁₂alicyclyl). The alicyclyl may be unsubstituted or substituted, and attached to the parent molecular moiety through any substitutable atom contained within the ring system.

The term “cycloalkyl” or “cycloalkane” as used herein, includes a monocyclic, a bicyclic, a tricyclic cycloalkyl, or higher polycyclic cycloalkyl ring. The monocyclic cycloalkyl is a carbocyclic ring system containing three to twelve carbon atoms and zero double bonds. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring, or a bridged monocyclic ring system in which two non-adjacent carbon atoms of the monocyclic ring are linked by an alkylene bridge containing one, two, three, or four carbon atoms. In some embodiments, the bicyclic cycloalkyl may have from seven to twenty-two carbon atoms. In other embodiments, the bicyclic cycloalkyl may have from seven to twelve carbon atoms. Representative examples of bicyclic cycloalkyls include, but are not limited to, bicyclo[3.1.1]heptyl, bicyclo[2.2.1]heptyl, bicyclo[2.2.2]octyl (including bicyclo[2.2.2]oct-1-yl), bicyclo[3.2.2]nonyl, bicyclo[3.3.1]nonyl, and bicyclo[4.2.1]nonyl. Tricyclic cycloalkyl refers to a bicyclic cycloalkyl fused to a monocyclic cycloalkyl, or a bicyclic cycloalkyl in which two non-adjacent carbon atoms of the ring system are linked by an alkylene bridge of between one and four carbon atoms of the bicyclic cycloalkyl ring. In some embodiments, tricyclic cycloalkyl may have from nine to thirty-two carbon atoms. In other embodiments, tricyclic cycloalkyl may have from nine to twelve carbon atoms. Higher polycyclic cycloalkyl rings include four or more rings. Representative examples of tricyclic-ring systems include, but are not limited to, tricyclo[3.3.1.0^{3,7}]nonane (octahydro-2,5-methanopentalene or noradamantane), and tricyclo[3.3.1.1^{3,7}]decane (adamantane). Higher polycyclic cycloalkyls include, for example, 3a,3b,4,6a,7,7a-hexahydro-3H-3,4,7-(epimethanetriyl)cyclopenta[a]pentalene and octahydro-1H-3,5,1-(epiethane[1,1,2]triyl)cyclobuta[cd]pentalene. The monocyclic, bicyclic, and tricyclic cycloalkyl may also form a spirocyclic ring system with an additional carbocyclic ring (e.g., spiro[5.5]undecane, octahydrospiro[cyclopropane-1,7′-[2,5]methanopentalene], spiro[bicyclo[3.3.1]nonane-9,1′-cyclopropane], spiro[adamantane-2,1′-cyclopropane]). The monocyclic, bicyclic, and tricyclic cycloalkyls may be unsubstituted or substituted, and are attached to the parent molecular moiety through any substitutable atom contained within the ring system.

The term “cycloalkenyl” or “cycloalkene” as used herein, means a monocyclic or a bicyclic non-aromatic hydrocarbon ring system. The monocyclic cycloalkenyl has four to twelve carbon atoms. Representative examples of monocyclic cycloalkenyl groups include, but are not limited to, cyclobutenyl, cyclopentenyl, cyclohexenyl, cycloheptenyl and cyclooctenyl. The bicyclic cycloalkenyl is a monocyclic cycloalkenyl fused to a monocyclic cycloalkyl group, a monocyclic cycloalkenyl fused to a monocyclic cycloalkenyl group, or a bridged monocyclic cycloalkenyl in which two non-adjacent carbon atoms of the monocyclic cycloalkenyl are linked by an alkylene bridge containing one, two, three, or four carbon atoms. Representative examples of the bicyclic cycloalkenyl groups include, but are not limited to, 4,5,6,7-tetrahydro-3aH-indene, octahydronaphthalene, bicyclo[2.2.2]oct-2-ene, and 1,6-dihydro-pentalene. The monocyclic and bicyclic cycloalkenyl may also form a spirocyclic ring system with an additional carbocyclic ring (e.g., spiro[5.5]undec-2-ene, spiro[bicyclo[2.2.2]octane-2,1′-cyclopropan]-5-ene). The monocyclic and bicyclic cycloalkenyl may be unsubstituted or substituted, and can be attached to the parent molecular moiety through any substitutable atom contained within the ring systems.

The term “heteroaryl,” as used herein, refers to an aromatic ring system containing at least one heteroatom selected from N, O, and S. A heteroaryl may be monocyclic, bicyclic, or tricyclic. Representative examples of monocyclic heteroaryl include, but are not limited to, furanyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl is an 8- to 12-membered ring system having a monocyclic heteroaryl fused to an additional ring; wherein the additional ring may be aromatic, saturated, or partially saturated, and may contain additional heteroatoms. Representative examples of bicyclic heteroaryl include, but are not limited to, benzofuranyl, benzoxadiazolyl, 1,3-benzothiazolyl, benzimidazolyl, benzodioxolyl, benzothienyl, chromenyl, furopyridinyl, indolyl, indazolyl, isoquinolinyl, naphthyridinyl, oxazolopyridine, quinolinyl, thienopyridinyl, 5,6,7,8-tetrahydroquinolinyl, 6,7-dihydro-5H-cyclopenta[b]pyridinyl, and 2,3-dihydrofuro[3,2-b]pyridinyl. The tricyclic heteroaryl is a 11- to 18-membered ring system having a bicyclic heteroaryl fused to an additional ring, wherein the additional ring may be aromatic, saturated, or partially saturated, and may contain additional heteroatoms. Representative examples of tricyclic heteroaryl include, but are not limited to, acridine, naphtho[2,3-b]thiophene, 9H-carbazole, dibenzo[b,d]thiophene, dibenzo[b,d]furan, and benzo[f]quinoline. The monocyclic, bicyclic, and tricyclic heteroaryl groups are connected to the parent molecular moiety through any substitutable carbon atom or any substitutable nitrogen atom contained within the groups.

A 5- or 6-membered nitrogen-containing heteroaryl contains at least one nitrogen ring atom and the other ring atoms are carbon, oxygen, nitrogen, or sulfur. Representative examples of 5-membered nitrogen-containing heteroaryl include, but are not limited to, pyrrolyl, imidazolyl, pyrazolyl, triazolyl, tetrazolyl, oxazolyl, isoxazolyl, thiazolyl, and isothiazolyl. Representative examples of 6-membered nitrogen-containing heteroaryl include, but are not limited to, pyridinyl, pyridazinyl, pyrimidinyl, and pyrazinyl. The 5- or 6-membered nitrogen-containing heteroaryl may be unsubstituted or substituted, and may be connected to the parent molecular moiety through any substitutable carbon atom or any substitutable nitrogen atom contained within the groups.

The term “heteroalicyclic” or “heteroalicycle” refers to an alicyclyl, wherein 1-3 ring atoms are independently replaced with O, N, or S. Included within alicyclyl are monocyclic, bicyclic, and tricyclic heterocycles, each of which may form a spirocyclic ring system with an additional carbocyclic or heterocyclic ring.

The term “heterocycle” or “heterocyclic” as used herein, refers to a non-aromatic ring system containing at least one heteroatom selected from N, O, and S. The heterocyclyl includes monocyclic, bicyclic, and tricyclic ring systems. The monocyclic heterocycle is a 3- to 12 membered ring system containing at least one heteroatom independently selected from the group consisting of 0, N, and S. Representative examples of monocyclic heterocycle include, but are not limited to, azetidinyl, azepanyl, aziridinyl, diazepanyl, 1,3-dioxanyl, 1,4-dioxanyl, 1,3-dioxolanyl, 4,5-dihydroisoxazol-5-yl, 3,4-dihydropyranyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl, thiopyranyl, and trithianyl. The bicyclic heterocycle is a 5-12-membered ring system having a monocyclic heterocycle fused to a phenyl, a saturated or partially saturated carbocyclic ring, or another monocyclic heterocyclic ring. The bicyclic heterocycle also includes a bridged monocyclic heterocycle in which two non-adjacent atoms (carbon or nitrogen) of the monocyclic heterocycle are linked by an alkylene bridge containing one, two, three, or four carbon atoms. Representative examples of bicyclic heterocycle include, but are not limited to, 3-azabicyclo[3.3.1]nonane, quinuclidine, 2-azabicyclo[2.2.1]heptane, 1,3-benzodioxol-4-yl, 1,3-benzodithiolyl, 3-azabicyclo[3.1.0]hexanyl, hexahydro-1H-furo[3,4-c]pyrrolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl. The tricyclic heterocycle is a bicyclic heterocycle fused to a phenyl, a bicyclic heterocycle fused to a monocyclic cycloalkyl, a bicyclic heterocycle fused to a monocyclic cycloalkenyl, or a bicyclic heterocycle fused to a monocyclic heterocycle. The tricyclic heterocycle also includes a bicyclic heterocycle in which two non-adjacent atoms of the ring system are linked by an alkylene bridge of between one and four carbon atoms of the bicyclic ring. Representative examples of tricyclic heterocycle include, but are not limited to, 2-oxatricyclo[3.3.1.1^{3,7}]decane, 2-azaadamantane, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl. The monocyclic, bicyclic, and tricyclic heterocycles may also form a spirocyclic ring system with an additional carbocyclic or heterocyclic ring. A representative example of a spirocyclic heterocycle is 3-azaspiro[5.5]undecane. The monocyclic, bicyclic, tricyclic, spirocyclic and bridged heterocycle groups are connected to the parent molecular moiety through any substitutable carbon atom or any substitutable nitrogen atom contained within the group. In some embodiments are 4- to 8-membered heterocycles that includes 4- to 8-membered monocyclic heterocycles and 5- to 8-membered bicyclic hetereocycles as described above.

A 4- to 8-membered nitrogen-containing heterocycle contains at least one nitrogen ring atom and optionally 1-2 additional heteroatoms selected from oxygen, nitrogen, and sulfur. Representative examples of 4- to 8-membered nitrogen-containing heterocycle include, but are not limited to, azetidinyl, pyrrolidinyl, piperidinyl, piperazinyl, imidazolinyl, imidazolidinyl, isothiazolinyl, isothiazolidinyl, isoxazolinyl, isoxazolidinyl, morpholinyl, oxazolinyl, oxazolidinyl, pyrazolinyl, pyrazolidinyl, pyrrolinyl, thiazolinyl, thiazolidinyl, and thiomorpholinyl. The 4- to 8-membered nitrogen-containing heterocycle may be unsubstituted or substituted, and is connected to the parent molecular moiety through any substitutable carbon atom or any substitutable nitrogen atom contained within the group.

The term “silacyclyl” or “silacycle” refers to an alicyclyl or heteroalicyclyl, wherein one or more ring carbon atoms are replaced by a silicon atom. In some embodiments, one ring atom is replaced by a silicon atom. Silacycles may also form spiro ring systems with additional carbocyclic or heterocyclic rings.

Terms such as “alkyl,” “cycloalkyl,” “alkylene,” etc. may be preceded by a designation indicating the number of atoms present in the group in a particular instance (e.g., “C₁₋₆alkyl,” “C₃₋₆cycloalkyl”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₆,” the members of the group that follows may have any number of carbon atoms falling within the recited range. A “C₁₋₆alkyl,” for example, is an alkyl group having from 1 to 6 carbon atoms, however arranged (i.e., straight chain or branched).

### Compounds

The present invention provides organo-transition metal complexes of formula (I) and compositions thereof, for use in the treatment of influenza. Generally, the compounds of formula (I) are composed of a transition metal M and its ligands L¹ and L². L¹ is made up of a derivatized M2 proton channel blocker moiety capable of complexing with a transition metal, such as those described herein. M2 proton channel blockers may be derivatized as described herein with groups —Y¹—X¹ to form various embodiments of L¹. M2 proton channel blockers are known in the art such as those described in:
Aldrich, P. E.; Hermann, E. C.; Meier, W. E.; Paulshock, M.; Prichard, W. W.; Snyder, J. A.; Watts, J. C., Antiviral agents. 2. Structure-activity relationships of compounds related to 1-adamantanamine. J Med Chem 1971, 14, 535-43.
Kolocouris, N.; Foscolos, G. B.; Kolocouris, A.; Marakos, P.; Pouli, N.; Fytas, G.; Ikeda, S.; De Clercq, E., Synthesis and antiviral activity evaluation of some aminoadamantane derivatives. J Med Chem 1994, 37, 2896-902.
Kolocouris, N.; Kolocouris, A.; Foscolos, G. B.; Fytas, G.; Neyts, J.; Padalko, E.; Balzarini, J.; Snoeck, R.; Andrei, G.; De Clercq, E., Synthesis and antiviral activity evaluation of some new aminoadamantane derivatives. 2. J Med Chem 1996, 39, 3307-18.
Kolocouris, A.; Tataridis, D.; Fytas, G.; Mavromoustakos, T.; Foscolos, G. B.; Kolocouris, N.; De Clercq, E., Synthesis of 2-(2-adamantyl)piperidines and structure anti-influenza virus A activity relationship study using a combination of NMR spectroscopy and molecular modeling. Bioorg Med Chem Lett 1999, 9, 3465-70.
Stamatiou, G.; Kolocouris, A.; Kolocouris, N.; Fytas, G.; Foscolos, G. B.; Neyts, J.; De Clercq, E., Novel 3-(2-adamantyl)pyrrolidines with potent activity against influenza A virus-identification of aminoadamantane derivatives bearing two pharmacophoric amine groups. Bioorg Med Chem Lett 2001, 11, 2137-42.
Stamatiou, G.; Foscolos, G. B.; Fytas, G.; Kolocouris, A.; Kolocouris, N.; Pannecouque, C.; Witvrouw, M.; Padalko, E.; Neyts, J.; De Clercq, E., Heterocyclic rimantadine analogues with antiviral activity. Bioorganic & Medicinal Chemistry 2003, 11, 5485-92.
Stylianakis, I.; Kolocouris, A.; Kolocouris, N.; Fytas, G.; Foscolos, G. B.; Padalko, E.; Neyts, J.; De Clercq, E., Spiro[pyrrolidine-2,2′-adamantanes]: synthesis, anti-influenza virus activity and conformational properties. Bioorg Med Chem Lett 2003, 13, 1699-703.
Zoidis, G.; Kolocouris, N.; Foscolos, G. B.; Kolocouris, A.; Fytas, G.; Karayannis, P.; Padalko, E.; Neyts, J.; De Clercq, E., Are the 2-isomers of the drug rimantadine active anti-influenza A agents? Antivir Chem Chemother 2003, 14, 153-64.
Setaki, D.; Tataridis, D.; Stamatiou, G.; Kolocouris, A.; Foscolos, G. B.; Fytas, G.; Kolocouris, N.; Padalko, E.; Neyts, J.; De Clercq, E., Synthesis, conformational characteristics and anti-influenza virus A activity of some 2-adamantylsubstituted azacycles. Bioorganic Chemistry 2006, 34, 248-73.
Zoidis, G.; Fytas, C.; Papanastasiou, I.; Foscolos, G. B.; Fytas, G.; Padalko, E.; De Clercq, E.; Naesens, L.; Neyts, J.; Kolocouris, N., Heterocyclic rimantadine analogues with antiviral activity. Bioorganic & Medicinal Chemistry 2006, 14, 3341-8.
Kolocouris, N.; Zoidis, G.; Foscolos, G. B.; Fytas, G.; Prathalingham, S. R.; Kelly, J. M.; Naesens, L.; De Clercq, E., Design and synthesis of bioactive adamantane spiro heterocycles. Bioorg Med Chem Lett 2007, 17, 4358-62.
Tataridis, D.; Fytas, G.; Kolocouris, A.; Fytas, C.; Kolocouris, N.; Foscolos, G. B.; Padalko, E.; Neyts, J.; De Clercq, E., Influence of an additional 2-amino substituent of the 1-aminoethyl pharmacophore group on the potency of rimantadine against influenza virus A. Bioorg Med Chem Lett 2007, 17, 692-6.
Kolocouris, A.; Spearpoint, P.; Martin, S. R.; Hay, A. J.; Lopez-Querol, M.; Sureda, F. X.; Padalko, E.; Neyts, J.; De Clercq, E., Comparisons of the influenza virus A M2 channel binding affinities, anti-influenza virus potencies and NMDA antagonistic activities of 2-alkyl-2-aminoadamantanes and analogues. Bioorg Med Chem Lett 2008, 18, 6156-60.
Zoidis, G.; Tsotinis, A.; Kolocouris, N.; Kelly, J. M.; Prathalingam, S. R.; Naesens, L.; De Clercq, E., Design and synthesis of bioactive 1,2-annulated adamantane derivatives. Organic & Biomolecular Chemistry 2008, 6, 3177-85.
Zoidis, G.; Kolocouris, N.; Naesens, L.; De Clercq, E., Design and synthesis of 1,2-annulated adamantane piperidines with anti-influenza virus activity. Bioorganic & Medicinal Chemistry 2009, 17, 1534-41.
Whitney, J. G.; Gregory, W. A.; Kauer, J. C.; Roland, J. R.; Snyder, J. A.; Benson, R. E.; Hermann, E. C., Antiviral agents. I. Bicyclo[2.2.2]octan-and-oct-2-enamines. J Med Chem 1970, 13, 254-60.
Fytas, G.; Marakos, P.; Kolocouris, N.; Foscolos, G. B.; Pouli, N.; Vamvakides, A.; Ikeda, S.; De Clercq, E., 3-Cyclopentyl-1-adamantanamines and adamantanemethanamines. Antiviral activity evaluation and convulsions studies. Farmaco 1994, 49, 641-7.
Tu, Q.; Pinto, L. H.; Luo, G.; Shaughnessy, M. A.; Mullaney, D.; Kurtz, S.; Krystal, M.; Lamb, R. A., Characterization of inhibition of M2 ion channel activity by BL-1743, an inhibitor of influenza A virus. Journal of virology 1996, 70, 4246-52.
Wang, J.; Cady, S. D.; Balannik, V.; Pinto, L. H.; DeGrado, W. F.; Hong, M., Discovery of spiro-piperidine inhibitors and their modulation of the dynamics of the M2 proton channel from influenza A virus. Journal of the American Chemical Society 2009, 131, 8066-76.
Wang, J.; Ma, C.; Balannik, V.; Pinto, L. H.; Lamb, R. A.; Degrado, W. F., Exploring the requirements for the hydrophobic scaffold and polar amine in inhibitors of M2 from Influenza A virus. ACS Med Chem Lett 2011, 2, 307-312.
Wang, J.; Ma, C.; Fiorin, G.; Carnevale, V.; Wang, T.; Hu, F.; Lamb, R. A.; Pinto, L. H.; Hong, M.; Klein, M. L.; DeGrado, W. F., Molecular dynamics simulation directed rational design of inhibitors targeting drug-resistant mutants of influenza A virus M2. J. Am. Chem. Soc. 2011, 133, 12834-12841.
Wang, J.; Ma, C.; Jo, H.; Canturk, B.; Fiorin, G.; Pinto, L. H.; Lamb, R. A.; Klein, M. L.; Degrado, W. F., Discovery of novel dual inhibitors of wild-type and the most prevalent drug-resistant mutant, S31N, of M2 proton channel from influenza A virus. J Med Chem 2013, 56, 2804-2812.
Wang, J.; Ma, C.; Wu, Y.; Lamb, R. A.; Pinto, L. H.; DeGrado, W. F., Exploring organosilane amines as potent inhibitors and structural probes of influenza A virus M2 proton channel. J Am. Chem. Soc. 2011, 133, 13844-13847.
Wang, J.; Wu, Y.; Ma, C.; Fiorin, G.; Pinto, L. H.; Lamb, R. A.; Klein, M. L.; Degrado, W. F., Structure and inhibition of the drug-resistant S31N mutant of the M2 ion channel of influenza A virus. Proceedings of the National Academy of Sciences of the U.S. Pat. No. 2,013,110, 1315-20.
Wu, Y.; Canturk, B.; Jo, H.; Ma, C.; Gianti, E.; Klein, M. L.; Pinto, L. H.; Lamb, R. A.; Fiorin, G.; Wang, J.; DeGrado, W. F., Flipping in the Pore: Discovery of Dual Inhibitors That Bind in Different Orientations to the Wild-Type versus the Amantadine-Resistant S31N Mutant of the Influenza A Virus M2 Proton Channel. Journal of the American Chemical Society 2014, 136, 17987-95.
Balgi, A. D.; Wang, J.; Cheng, D. Y.; Ma, C.; Pfeifer, T. A.; Shimizu, Y.; Anderson, H. J.; Pinto, L. H.; Lamb, R. A.; Degrado, W. F.; Roberge, M., Inhibitors of the influenza a virus m2 proton channel discovered using a high-throughput yeast growth restoration assay. PLoS One 2013, 8, e55271.
Mao, L.; Wang, J.; Degrado, W. F.; Inouye, M., An assay suitable for high throughput screening of anti-influenza drugs. PLoS One 2013, 8, e54070.
Rey-Carrizo, M.; Torres, E.; Ma, C.; Barniol-Xicota, M.; Wang, J.; Wu, Y.; Naesens, L.; DeGrado, W. F.; Lamb, R. A.; Pinto, L. H.; Vazquez, S., 3-Azatetracyclo[5.2.1.1(5,8).0(1,5)]undecane derivatives: from wild-type inhibitors of the M2 ion channel of influenza A virus to derivatives with potent activity against the V27 Å mutant. J Med Chem 2013, 56, 9265-74.
Rey-Carrizo, M.; Barniol-Xicota, M.; Ma, C.; Frigole-Vivas, M.; Torres, E.; Naesens, L.; Llabres, S.; Juarez-Jimenez, J.; Luque, F. J.; DeGrado, W. F.; Lamb, R. A.; Pinto, L. H.; Vazquez, S., Easily accessible polycyclic amines that inhibit the wild-type and amantadine-resistant mutants of the M2 channel of influenza A virus. J Med Chem 2014, 57, 5738-47.
Kolocouris, A.; Hansen, R. K.; Broadhurst, R. W., Interaction between an amantadine analogue and the transmembrane portion of the influenza A M2 protein in liposomes probed by 1H NMR spectroscopy of the ligand. J Med Chem 2004, 47, 4975-8.
Zoidis, G.; Kolocouris, N.; Kelly, J. M.; Prathalingam, S. R.; Naesens, L.; De Clercq, E., Design and synthesis of bioactive adamantanaminoalcohols and adamantanamines. Eur J Med Chem 2010, 45, 5022-30.
Kolocouris, A.; Tzitzoglaki, C.; Johnson, F. B.; Zell, R.; Wright, A. K.; Cross, T. A.; Tietjen, I.; Fedida, D.; Busath, D. D., Aminoadamantanes with persistent in vitro efficacy against H1N1 (2009) influenza A. J Med Chem 2014, 57, 4629-39.
Zhao, X.; Li, C.; Zeng, S.; Hu, W., Discovery of highly potent agents against influenza A virus. Eur J Med Chem 2011, 46, 52-7.
Zhao, X.; Jie, Y.; Rosenberg, M. R.; Wan, J.; Zeng, S.; Cui, W.; Xiao, Y.; Li, Z.; Tu, Z.; Casarotto, M. G.; Hu, W., Design and synthesis of pinanamine derivatives as anti-influenza A M2 ion channel inhibitors. Antiviral Res 2012, 96, 91-9.
Shibnev, V. A.; Garayev, T. M.; Finogenova, M. P.; Shevchenko, E. S.; Burtseva, E. I., [New adamantane derivatives and ways of overcoming the resistance of influenza A viruses to rimantadine and amantadine]. Vopr Virusol 2011, 56, 36-9.
Shibnev, V. A.; Garaev, T. M.; Finogenova, M. P.; Shevchenko, E. S.; Burtseva, E. I., New adamantane derivatives can overcome resistance of influenza A(H1N1)pdm2009 and A(H3N2) viruses to remantadine. Bulletin of experimental biology and medicine 2012, 153, 233-5.
Rubessa, F.; Runti, C.; Ulian, F.; Vio, L.; Zonta, F., [Antiviral agents. XIX. Amantadine derivatives and several others]. Il Farmaco; edizione scientifica 1977, 32, 129-40.
Indulen, M. K.; Polis, J. J.; Kalnina, V. A.; Ryasantzeva, G. M.; Dsegudse, D. R.; Eglite, F. E.; Zamyatina, N. A.; Kanninokova, N. A.; Feldblum, F. L., Antiviral activity of new amantadine derivatives. In Antiviral Activity and Mode of Action of Chemical Compounds., Kukaine, R. A.; Muszenieze, A. J.; Indulen, M. K.; Duk, A. E., Eds.: Sinatne, Riga (in Russian), 1979; pp 41-48.
Cheetham, J. J.; Epand, R. M., Comparison of the interaction of the anti-viral chemotherapeutic agents amantadine and tromantadine with model phospholipid membranes. Bioscience reports 1987, 7, 225-30.
Serkedjieva, J.; Zgorniak-Nowosielska, I., Combined antiinfluenza activity of a plant preparation SHS-174 and amantadine derivatives. Acta virologica 1993, 37, 258-64.
Serkedjieva, J., Combined antiinfluenza virus activity of Flos verbasci infusion and amantadine derivatives. Phytotherapy research: PTR 2000, 14, 571-4.
Fleming, D. M., Managing influenza: amantadine, rimantadine and beyond. Int J Clin Pract 2001, 55, 189-95.
Camps, P.; Duque, M. D.; Vazquez, S.; Naesens, L.; De Clercq, E.; Sureda, F. X.; Lopez-Querol, M.; Camins, A.; Pallas, M.; Prathalingam, S. R.; Kelly, J. M.; Romero, V.; Ivorra, D.; Cortes, D., Synthesis and pharmacological evaluation of several ring-contracted amantadine analogs. Bioorganic & medicinal chemistry 2008, 16, 9925-36.
Torres, E.; Fernandez, R.; Miquet, S. p.; Font-Bardia, M.; Vanderlinden, E.; Naesens, L.; Vazquez, S., Synthesis and anti-influenza A virus activity of 2,2-dialkylamantadines and related compounds. ACS Med Chem Lett 2012, 3, 1065-1069.
Bergstrom, D. E.; Abrahamson, J. K.; Chan, M. Y., Nucleoside and nucleotide transport through a model liquid membrane. Periodic-catastrophic transport of a novel amantadine phosphoramidate conjugate of 5′-AMP. Biochimica et biophysica acta 1991, 1061, 95-105.
Warnick, J. E.; Maleque, M. A.; Albuquerque, E. X., Interaction of bicyclo-octane analogs of amantadine with ionic channels of the nicotinic acetylcholine receptor and electrically excitable membrane. The Journal of pharmacology and experimental therapeutics 1984, 228, 73-9.
Rey-Carrizo, M.; Gazzarrini, S.; Llabres, S.; Frigole-Vivas, M.; Juarez-Jimenez, J.; Font-Bardia, M.; Naesens, L.; Moroni, A.; Luque, F. J.; Vazquez, S., New polycyclic dual inhibitors of the wild type and the V27 Å mutant M2 channel of the influenza A virus with unexpected binding mode. Eur J Med Chem 2015, 96, 318-29.
Torres, E.; Leiva, R.; Gazzarrini, S.; Rey-Carrizo, M.; Frigole-Vivas, M.; Moroni, A.; Naesens, L.; Vazquez, S., Azapropellanes with anti-influenza a virus activity. ACS Med Chem Lett 2014, 5, 831-6.
Duque, M. D.; Ma, C.; Torres, E.; Wang, J.; Naesens, L.; Juarez-Jimenez, J.; Camps, P.; Luque, F. J.; DeGrado, W. F.; Lamb, R. A.; Pinto, L. H.; Vazquez, S., Exploring the size limit of templates for inhibitors of the M2 ion channel of influenza A virus. J Med Chem 2011, 54, 2646-57.
Torres, E.; Duque, M. D.; Lopez-Querol, M.; Taylor, M. C.; Naesens, L.; Ma, C.; Pinto, L. H.; Sureda, F. X.; Kelly, J. M.; Vazquez, S., Synthesis of benzopolycyclic cage amines: NMDA receptor antagonist, trypanocidal and antiviral activities. Bioorganic & medicinal chemistry 2012, 20, 942-8.
Torres, E.; Duque, M. D.; Vanderlinden, E.; Ma, C.; Pinto, L. H.; Camps, P.; Froeyen, M.; Vazquez, S.; Naesens, L., Role of the viral hemagglutinin in the anti-influenza virus activity of newly synthesized polycyclic amine compounds. Antiviral Res 2013, 99, 281-291.
Scholtissek, C.; Quack, G.; Klenk, H. D.; Webster, R. G., How to overcome resistance of influenza A viruses against adamantane derivatives. Antiviral Res 1998, 37, 83-95.
Varvaresou, A.; Iakovou, K.; Filippatos, E.; Souli, C.; Calogeropoulou, T.; Ioannidou, I.; Kourounakis, A. P.; Pannecouque, C.; Witvrouw, M.; Padalko, E.; Neyts, J.; De Clercq, E.; Tsotinis, A., Synthesis, antiretroviral and antioxidant evaluation of a series of new benzo[b]furan derivatives. Arzneimittel-Forschung 2001, 51, 156-62.
Gmiro, V. E.; Serdiuk, S. E., [Comparative study of the NMDA-blocking activity and safety of mono- and bis-cationic compounds in animals]. Eksperimental'naia i klinicheskaia farmakologiia 2000, 63, 3-8.
Balannik, V.; Wang, J.; Ohigashi, Y.; Jing, X.; Magavern, E.; Lamb, R. A.; Degrado, W. F.; Pinto, L. H., Design and pharmacological characterization of inhibitors of amantadine-resistant mutants of the M2 ion channel of influenza A virus. Biochemistry 2009, 48, 11872-82.
Kauer, J. C. Bicyclo[2.2.2]octane-1-amines and bicyclo[2.2.2]octane-1-methylamines. US003546290.
Sulli, C.; Banik, S. S. R.; Schilling, J.; Moser, A.; Xiang, X.; Payne, R.; Wanless, A.; Willis, S. H.; Paes, C.; Rucker, J. B.; Doranz, B. J., Detection of proton movement directly across viral membranes to identify novel influenza M2 inhibitors J Virology 2013, 87, 10679-10686.
Buchbauer, G.; Haslinger, E.; Robien, W.; Stefanich, M., [A new amantadine analog]. Archiv der Pharmazie 1985, 318, 631-4.
DeGrado, W. F.; Wang, J. Inhibitors of the influenza A virus M2 proton channel. U.S. Pat. No. 8,569,284.
DeGrado, W. F.; Wang, J. Spiro-piperidine inhibitors. U.S. Pat. No. 8,557,836 B2.
Tonew, M.; Tonew, E.; Indulen, M. K.; Dzeguze, D. R.; Oehring, H.; Schmidt, J., Antiviral activity of hetarylhydrazones. Acta virologica 1982, 26, 130-6.
Indulen, M. K.; Polis, J. J.; Kanele, I. A.; Ryasantzeva, G. M.; Dsegudse, D. R.; Kalnina, V. A.; Grabe, I. J., Antiviral activity of a series of rimantadine derivatives. In Antiviral Activity and Mode of Action of Chemical Compounds, Kukaine, R. A.; Muszenieze, A. J.; Indulen, M. K.; Duk, A. E., Eds.: Sinatne, Riga, 1979; pp 34-40.
Alhadeff, R.; Assa, D.; Astrahan, P.; Krugliak, M.; Arkin, I. T., Computational and experimental analysis of drug binding to the Influenza M2 channel. Biochimica et biophysica acta 2014, 1838, 1068-73.
Hu, W.; Zeng, S.; Li, C.; Jie, Y.; Li, Z.; Chen, L., Identification of hits as matrix-2 protein inhibitors through the focused screening of a small primary amine library. J Med Chem 2010, 53, 3831-4.
Ackermann, W. W.; Maassab, H. F., Growth characteristics of influenza virus: the influence of a sulfonic acid. The Journal of experimental medicine 1954, 99, 105-17.
Cochran, K. W.; Maassab, H. F.; Tsunoda, A.; Berlin, B. S., Studies on the antiviral activity of amantadine hydrochloride. Annals of the New York Academy of Sciences 1965, 130, 432-9.
Sidwell, R. W.; Dixon, G. J.; Sellers, S. M.; Schabel, F. M., Jr., In vivo antiviral properties of biologically active compounds. II. Studies with influenza and vaccinia viruses. Applied microbiology 1968, 16, 370-92.
Lundahl, K.; Schut, J.; Schlatmann, J. L.; Paerels, G. B.; Peters, A., Synthesis and antiviral activities of adamantane spiro compounds. I. Adamantane and analogous spiro-3′-pyrrolidines. J Med Chem 1972, 15, 129-32.
van Hes, R.; Smit, A.; Kralt, T.; Peters, A., Synthesis and antiviral activities of adamantane spiro compounds. 2. J Med Chem 1972, 15, 132-6.
Runti, C.; Rubessa, F., [Antiviral chemotherapeutic agents. XVII. Potential interferon inducers: adamantane derivatives]. Il Farmaco; edizione scientifica 1974, 29, 820-34.
Kanao, S.; Toyoda, T.; Suyama, T.; Toyoshima, S., [Syntheses of aminoacid derivatives and their biological activities. I. Anti-influenza activity (author's transl)]. Yakugaku zasshi: Journal of the Pharmaceutical Society of Japan 1975, 95, 397-401.
Mathur, A.; Beare, A. S.; Reed, S. E., In Vitro Antiviral Activity and Preliminary Clinical Trials of a New Adamantane Compound. Antimicrob Agents Chemother 1973, 4, 421-6.
Zarubaev, V. V.; Anfimov, P. M.; Shtro, A. A.; Garshinina, A. V.; Meleshkina, I. A.; Karpinskaia, L. A.; Kozeletskaia, K. N.; Kiselev, O. I., [Development of novel drugs against influenza virus based on synthetic and natural compounds]. Vopr Virusol 2012, 57, 30-6.
Kiselev, O. I.; Blinov, V. M.; Kozeletskaia, K. N.; Il'enko, V. I.; Platonov, V. G.; Chupakhin, O. N.; Stukova, M. A.; Karginov, V. A., [The molecular mechanism of the action of antiviral preparations in the adamantane series]. Vestnik Rossiiskoi akademii meditsinskikh nauk/Rossiiskaia akademiia meditsinskikh nauk 1993, 10-5.
Tilley, J. W.; Levitan, P.; Kramer, M. J., Adamantylthiourea derivatives as antiviral agents. J Med Chem 1979, 22, 1009-10.
Zarubaev, V. V.; Golod, E. L.; Anfimov, P. M.; Shtro, A. A.; Saraev, V. V.; Gavrilov, A. S.; Logvinov, A. V.; Kiselev, O. I., Synthesis and anti-viral activity of azolo-adamantanes against influenza A virus. Bioorganic & medicinal chemistry 2010, 18, 839-48.
Manchand, P. S.; Cerruti, R. L.; Martin, J. A.; Hill, C. H.; Merrett, J. H.; Keech, E.; Belshe, R. B.; Connell, E. V.; Sim, I. S., Synthesis and antiviral activity of metabolites of rimantadine. J Med Chem 1990, 33, 1992-5.
Fytas, C.; Kolocouris, A.; Fytas, G.; Zoidis, G.; Valmas, C.; Basler, C. F., Influence of an additional amino group on the potency of aminoadamantanes against influenza virus A. II-Synthesis of spiropiperazines and in vitro activity against influenza A H3N2 virus. Bioorganic chemistry 2010, 38, 247-51.
Lin, C.-H.; Chang, T.-T.; Sun, M.-F.; Chen, H.-Y.; Tsai, F.-J.; Chang, K.-L.; Fisher, M.; Chen, C. Y.-C., Potent inhibitor design against H1N1 swine influenza: structure-based and molecular dynamics analysis for M2 inhibitors from traditional Chinese medicine database. Journal of biomolecular structure & dynamics 2011, 28, 471-82.
Tran, L.; Choi, S. B.; Al-Najjar, B. O.; Yusuf, M.; Wahab, H. A.; Le, L., Discovery of Potential M2 Channel Inhibitors Based on the Amantadine Scaffold via Virtual Screening and Pharmacophore Modeling. Molecules (Basel, Switzerland) 2011, 16, 10227-10255.
Li, C.; Long, Y.; Lin, Z.; Jie, Y.; Xiao, Y.; Yang, L.; Sun, J.; Ren, Y.; Chen, L.; Li, Z., New strategy for high throughput screening of anti-Influenza virus M2 ion channel inhibitors. Current pharmaceutical design 2013.
   Each of the foregoing references is incorporated herein by reference in its entirety.

In another aspect of the invention are provided compounds of formula (I), or salts thereof, wherein

M is a transition metal, where p is an integer from 0 to 5;

each L¹ is independently a derivative of an M2 proton channel blocker capable of complexing with M, L¹ being a bidentate or tridentate ligand;

each L² is independently an auxiliary ligand, L² being a monodentate, bidentate, tridentate, or tetradentate ligand;
m is 1, 2, or 3; and

n is an integer from 0 to 4;

In some embodiments, compounds of formula (I) include a proviso that excludes:
diaqua(N-(1-adamantyl)-iminodiacetate)copper(II);
bis-[(imidazole)(N-(1-adamantyl)-iminodiacetate)copper(II)];
(2,2′-bipyridine)(N-(1-adamantyl)-iminodiacetate)copper(II);
((1S,2S,3S,5R)-2,6,6-trimethyl-N-((1-methyl-1H-imidazol-2-yl)methyl)-bicyclo[3.1.1]heptan-3-amine)copper(II)diacetate or hydrate or solvate thereof;
and
((1 S,2 S,3 S,5R)-2,6,6-trimethyl-N-((1-methyl-1H-imidazol-2-yl)methyl)-bicyclo[3.1.1]heptan-3-amine)copper(II)dichloride or hydrate or solvate thereof.

In some embodiments, L¹ is a bidentate ligand. In other embodiments, L¹ is a tridentate ligand. In some embodiments, L¹ comprises a derivative of an M2 proton channel blocker capable of complexing with M. In some embodiments, L¹ comprises an M2 proton channel blocker moiety attached to an appendage having a transition metal-binding moiety. In some embodiments, L¹ comprises an M2 proton channel blocker moiety attached to one or two appendages, each independently having a metal-binding moiety (e.g., X¹). In some embodiments, the appendage comprises a metal-binding moiety and a linker (e.g., Y¹), the linker connecting the M2 proton channel blocker moiety to the metal-binding moiety.

As is understood by one skilled in the art, the number of L¹ and L² groups coordinated to M, and therefore the variables m and n, may vary depending on the specific ligands, the metal, and the metal oxidation state. In some embodiments, the coordination number of the metal is 6. In other embodiments, the coordination number is 5. In still other embodiments, the coordination number is 4. In yet other embodiments, the coordination number is an integer from 4-6. In some embodiments, M is Cu, p is 2, and the coordination number is 4 to 6. In other embodiments, M is Cu, p is 1, and the coordination number is 4. In other embodiments, M is Zn, p is 2, and the coordination number is 5 or 6. In other embodiments, M is Ni, p is 2, and the coordination number is 4 to 6.

In some embodiments, M is Cu, Zn, Ni, Co, Fe, Mn, Cr, V, Ti, Ag, Pd, Rh, Ru, Mo, Au, Pt, Ir, or W. In other embodiments, M is Cu, Zn, Ni or Co. In still other embodiments M is Cu, Zn, or Ni. In yet further embodiments, M is Cu.

In some embodiments, p is 0 and M is Pd or Pt. In some embodiments, p is 1 and M is Cu, Ag, Rh, Au, or Ir. In other embodiments, p is 2 and M is Cu, Zn, Ni, Co, Fe, Mn, Cr, V, Ti, Pd, Ru, or Pt. In other embodiments, p is 2 and M is Cu, Zn, Ni, or Co. In certain embodiments, p is 2 and M is Cu. In still other embodiments, p is 3 and M is Co, Fe, Mn, Cr, V, Rh, Ru, Mo, Au, Ir, or W. In still other embodiments, p is 4 and M is Ti, Pd, Pt, or W. In yet other embodiments, p is 5 and M is V.

In some embodiments, G¹ is an alicyclyl, the alicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy. In some embodiments, the alicyclyl at G¹ is unsubstituted. In some embodiments, the alicyclyl has from three to thirty-two carbon ring atoms, i.e., C₃₋₃₂alicyclyl. In other embodiments, the alicyclyl has from three to sixteen carbon ring atoms, i.e., C₃₋₁₆alicyclyl. In other embodiments, the alicyclyl has from three to twelve carbon ring atoms, i.e., C₃₋₁₂alicyclyl. In other embodiments, the alicyclyl has from three to ten carbon ring atoms, i.e., C₃₋₁₀alicyclyl. In other embodiments, the alicyclyl has from six to twelve carbon ring atoms (C₆₋₁₂alicyclyl). In some embodiments, the alicyclyl at G¹ is selected from the group consisting of a monocyclic cycloalkyl (e.g., cyclooctyl), a monocyclic cycloalkenyl (e.g., cyclooctenyl), a bicyclic cycloalkyl (e.g., bicyclo[2.2.2]octane, bicyclo[2.2.1]heptane), a bicyclic cycloalkenyl (e.g., bicyclo[2.2.2]oct-2-ene), a tricyclic cycloalkyl (e.g., adamantane, noradamantane, tricyclo[3.3.0.0^{3,7}]octane, 1,5-dimethyltricyclo[3.3.0.0^{3,7}]octane, octahydro-2,5-methanopentalene), or a higher polycyclic cycloalkyl (e.g., octahydro-1H-3,5,1-(epiethane[1,1,2]triyl)cyclobuta[cd]pentalen-2-yl, 3a,3b,4,6a,7,7a-hexahydro-3H-3,4,7-(epimethanetriyl)cyclopenta[a]pentalen-8-yl), the monocyclic cycloalkyl, the monocyclic cycloalkenyl, the bicyclic cycloalkyl, the bicyclic cycloalkenyl, the tricyclic cycloalkyl, and the higher polycyclic cycloalkyl being optionally joined to a second alicyclic ring to form a spirocyclic ring system (e.g., spiro[5.5]undecane, spiro[6.6]tridecane, spiro[adamantane-2,1′-cyclopropane]) and G¹ is optionally substituted as defined herein. In some embodiments, G¹ is cyclooctyl, 2,6,6-trimethylbicyclo[3.1.1]heptan-3-yl, spiro[5.5]undecan-3-yl, or adamant-1-yl.

In some embodiments, G¹ is a heteroalicyclyl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₁₀haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀haloalkyl, halo, C₁₋₆alkoxy, and C₁₋₆haloalkoxy. In some embodiments, the heteroalicyclyl at G¹ is selected from a monocyclic heterocycle (e.g., pyrrolidine, piperidine), a bicyclic heterocycle (e.g., quinuclidine, 2-azabicyclo[2.2.1]heptane), or a tricyclic heterocycle (2-oxatricyclo[3.3.1.1^{3,7}]decane), the monocyclic, bicyclic, and tricyclic heterocycle being optionally joined to an additional carbocyclic or heterocyclic ring to form a spiro ring system (e.g., 3-azaspiro[5.5]undecane).

In some embodiments, G¹ is a silacycle optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₁₀ haloalkoxy. In some embodiments, the silacycle at G¹ is a monocyclic silacycle (e.g., 1,1-dimethylsilinane, 4,4-dimethyl-1,4-azasilepan-1-yl), the monocyclic silacycle being optionally joined to an additional ring to form a spiro ring system (e.g., 6-silaspiro[5.5]undecane, 5-silaspiro[4.5]decane, 8-aza-5-silaspiro[4.6]undecan-8-yl).

In some embodiments, G¹ is a C₆₋₂₀aryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy. In some embodiments, G¹ is a monocyclic aryl (i.e., phenyl), a bicyclic aryl (e.g., naphthyl, indanyl), or a tricyclic aryl (e.g., 9H-fluoren-9-one, anthracenyl, phenanthrenyl, 9-methyl-5,6,8,9,10,11-hexahydro-7H-5,9:7,11-dimethanobenzo[9]annulen-7-yl), the monocyclic, bicyclic, and tricyclic aryl being optionally substituted as defined herein.

In some embodiments, G¹ is a 5- to 20-membered heteroaryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy. In some embodiments, G¹ is a monocyclic heteroaryl (e.g., pyridine, pyrazine), a bicyclic heteroaryl (e.g., quinolone, indole), or a tricyclic heteroaryl (e.g., acridine, naphtho[2,3-b]thiophene, 9H-carbazole, dibenzo[b,d]thiophene, dibenzo[b,d]furan, and benzo[f]quinolone), the monocyclic, bicyclic, and tricyclic heteroaryl being optionally substituted as defined herein.

In some embodiments, each X¹ is independently OH, OC₁₋₄alkyl, NH₂, NH(C₁₋₄alkyl), N(C₁₋₄alkyl)(C₁₋₄alkyl), COOH, CONH₂, CONH(C₁₋₄alkyl), CON(C₁₋₄alkyl)(C₁₋₄alkyl), C(NH)NH₂, NHC(NH)NH₂, NHOH, SH, S(C₁₋₄alkyl), C(NC₁₋₄alkyl), a 5- or 6-membered nitrogen-containing heteroaryl (e.g., 1H-pyrrol-2-yl, pyrazol-5-yl, 1H-imidazol-2-yl, 1H-imidazol-4-yl, 1H-1,2,3-triazol-4-yl, isoxazol-3-yl, pyridine-2-yl), or a 4- to 8-membered nitrogen-containing heterocycle (e.g., azetidin-2-yl, pyrrolidin-2-yl, piperidin-2-yl, etc.), or salts thereof, the 5- or 6-membered nitrogen-containing heteroaryl and the 4- to 8-membered nitrogen-containing heterocycle each being independently optionally substituted with 1-4 substituents independently selected from the group consisting of C₁₋₄alkyl, C₁₋₄haloalkyl, halo, C₁₋₄alkoxy, and C₁₋₄haloalkoxy. Salts of the listed X¹ group members include, for example, a carboxylate salt and a salt of a tetrazole moiety. In other embodiments, each X¹ is independently NH₂, COOH, CONH₂, 1-methyl-1H-imidazol-2-yl, or salts thereof (e.g., carboxylate ion). Where two or more X¹ are present, the X¹ may be the same or different.

In some embodiments, Y¹—X¹ is independently selected from the group consisting of

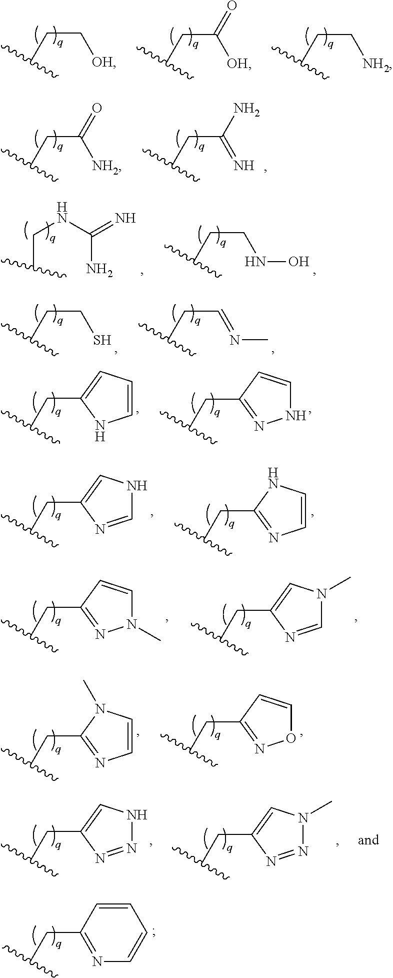

and q is 1 or 2. In other embodiments, Y¹—X¹ is selected from the group consisting of —CH₂CH₂NH₂, —CH₂COOH, —CH₂CONH₂, and (1-methyl-1H-imidazol-2-yl)methyl, or salts thereof. Where two or more Y¹—X¹ are present, the Y¹—X¹ may be the same or different.

In some embodiments, L¹ is G¹-Y²—N(R¹)—Y¹—X¹, wherein G¹, Y¹, and X¹ are as defined herein.

In some embodiments, —Y¹—X¹ is defined as in the embodiments above, G¹ is alicyclic, and G¹-Y²—N(R¹)— is selected from:

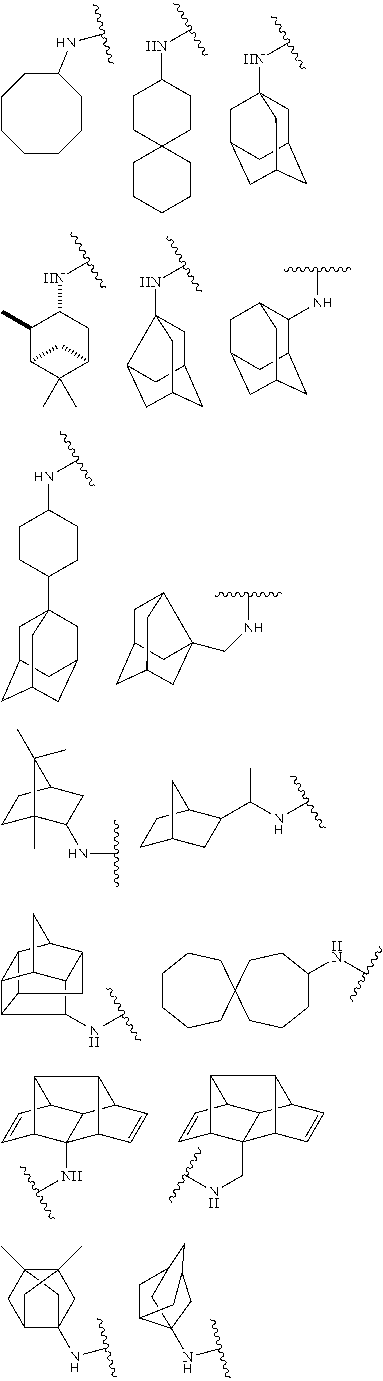

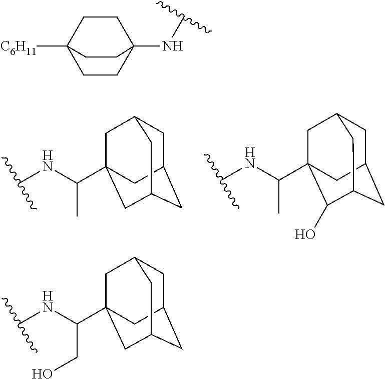

In some embodiments, —Y¹—X¹ is defined as in the embodiments above, G¹ is alicyclic, and G¹-Y²—N(R¹)— is selected from:

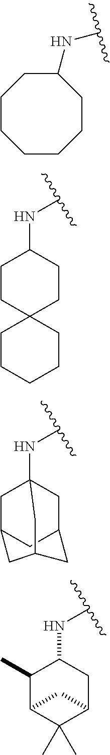

In some embodiments, —Y¹—X¹ is defined as in the embodiments above, G¹ is heteroalicyclic, and G¹-Y²—N(R¹)— is selected from:

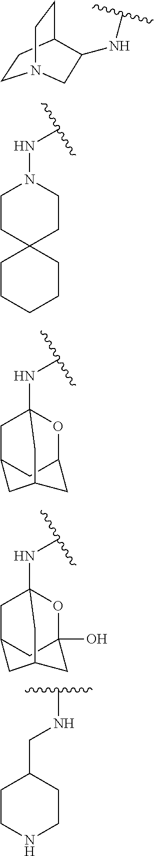

In some embodiments, Y¹—X¹ is defined as in the embodiments above, G¹ is silacyclyl, and G¹-Y²—N(R¹)— is selected from:

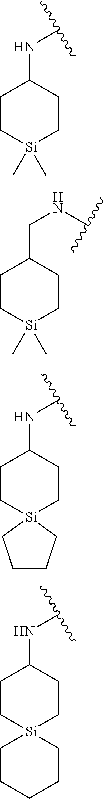

In some embodiments, Y¹—X¹ is defined as in the embodiments above, G¹ is C₆₋₂₀aryl, and G¹-Y²—N(R¹)— is selected from:

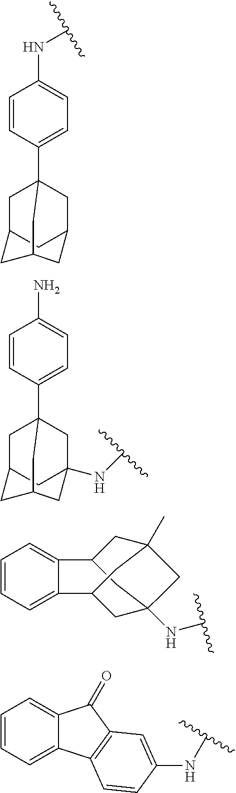

In some embodiments, Y¹—X¹ is defined as in the embodiments above, G¹ is a 5- to 20-membered heteroaryl and G¹-Y²—N(R¹)— is selected from:

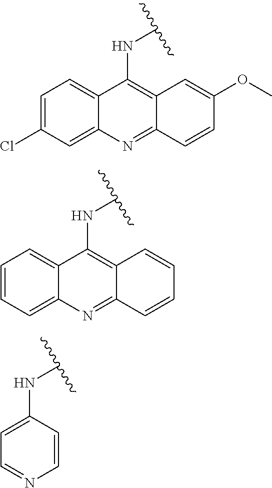 [SMILES: *Nc1c2ccc(Cl)cc2nc2ccc(OC)cc12 ; *Nc1c2ccccc2nc2ccccc12 ; *Nc1ccncc1]

In some embodiments, L¹ is independently G¹-Y²—N(Y¹—X¹)₂, wherein G¹, Y², Y¹, and X¹ are as defined herein. In embodiments where L¹ is G¹-Y²—N(Y¹—X¹)₂, G¹ may be selected as set forth above for the embodiments wherein L¹ is G¹-Y²—N(R¹)—Y¹—X¹, by further substitution of a second group Y¹—X¹ on the nitrogen atom between Y¹ and Y². In some embodiments, —Y¹—X¹ is defined as in the embodiments above, and G¹-Y²—N is selected from:

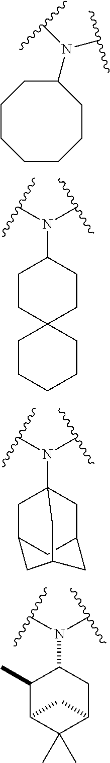

In the foregoing embodiments where L¹ is G¹-Y²—N(Y¹—X¹)₂, the —Y¹—X¹ may be the same or different.

In some embodiments, L¹ is G²(-Y¹—X¹)ᵣ, wherein G², Y¹, X¹, and r are as defined herein. In some embodiments, G² is a heteroalicyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy. In some embodiments, Y¹—X¹ is attached to the ring nitrogen of the heteroalicyclyl of G². In some embodiments, Y¹—X¹ is defined as in the embodiments above and G² is selected from:

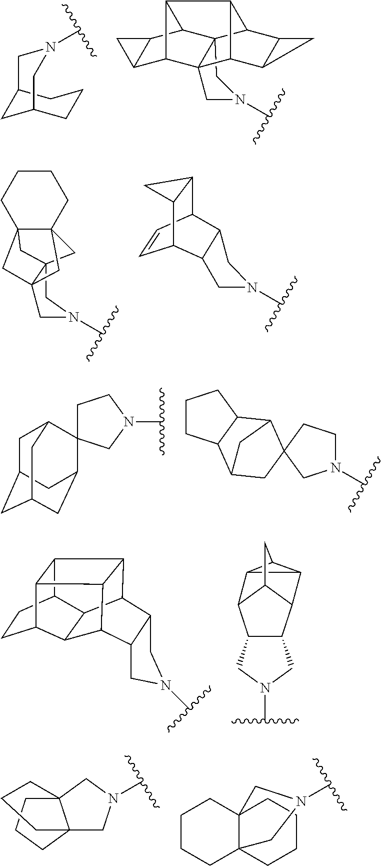

In some embodiments, G² is a silacyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy. In some embodiments, —Y¹—X¹ is attached to the ring nitrogen of the silacyclyl of G². In some embodiments, —Y¹—X¹ is defined as in the embodiments above and G² is selected from:

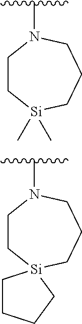

Where two or more L¹ are present, the L¹, as described herein, may be the same or different.

Each L² is independently an auxiliary ligand, L² being a monodentate, bidentate, tridentate, or tetradentate ligand. L² includes, but is not limited to, water, pyridine, a halide ion, cyanide ion, an acetate ion, phosphate ion, sulfate ion, carbonate ion, bicarbonate ion, nitrate ion,

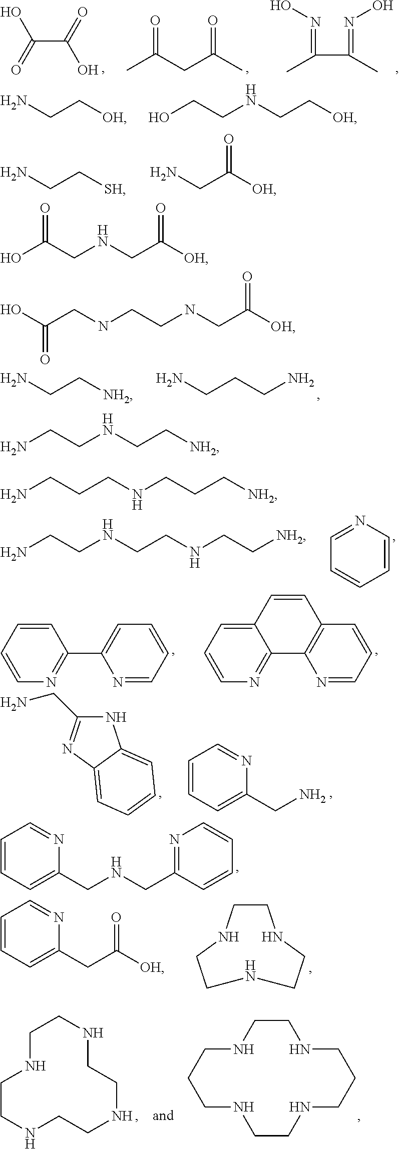

or salts thereof

Compounds described herein may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30.

The various stereoisomers (including enantiomers and diastereomers) and mixtures thereof of the compounds described are also contemplated. Metal complexes of the invention may exist as stereoisomers. Individual stereoisomers of compounds described may be prepared synthetically from commercially available starting materials that contain asymmetric or chiral centers or by preparation of racemic mixtures followed by resolution of the individual stereoisomer using methods that are known to those of ordinary skill in the art. Examples of resolution are, for example, (i) attachment of a mixture of enantiomers to a chiral auxiliary, separation of the resulting mixture of diastereomers by recrystallization or chromatography, followed by liberation of the optically pure product; or (ii) separation of the mixture of enantiomers or diastereomers on chiral chromatographic columns.

Geometric isomers may exist in the present compounds. Specifically, metal complexes of the invention may exist as stereoisomers. All various geometric isomers and mixtures thereof resulting from the disposition of substituents around a multiple bond (e.g., carbon-carbon double bond, a carbon-nitrogen double bond, a cycloalkyl group, or a heterocycle group) are contemplated. Substituents around a carbon-carbon double bond or a carbon-nitrogen bond are designated as being of Z or E configuration and substituents around a cycloalkyl or a heterocycle are designated as being of cis or trans configuration.

It is to be understood that compounds disclosed herein may exhibit the phenomenon of tautomerism.

Thus, the formulae within this specification can represent only one of the possible tautomeric forms. It is to be understood that encompassed herein are any tautomeric form, and mixtures thereof, and is not to be limited merely to any one tautomeric form utilized within the naming of the compounds or formulae.

Additionally, unless otherwise stated, the structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention.

Also contemplated as part of the invention are compounds formed by synthetic means or formed in vivo by biotransformation or by chemical means. For example, certain compounds of the invention may function as prodrugs that are converted to other compounds of the invention upon administration to a subject. For example, ligands L² may be replaced by water or physiological anions on exposure of compounds of formula (I) to biological fluids.

### Methods of Treatment

The compounds of formula (I) are active against the influenza A virus making the compounds and pharmaceutical compositions useful for treating influenza A virus infections. Included in the method of treatment is therapeutic treatment of a symptom, condition or disease caused by or associated with an influenza A virus infection. The compounds of formula (I) may inhibit either wild-type or S31N-bearing strains of influenza A. The condition or disease to be prevented, treated or alleviated is selected primarily from the group consisting of acute bronchitis, chronic bronchitis, rhinitis, sinusitis, croup, acute bronchiolitis, pharyngitis, tonsillitis, laryngitis, tracheitis, asthma and pneumonia and including typical symptoms frequently accompanying said conditions or diseases such as fever, pain, dizziness, shivering; sweating, and dehydration.

In another embodiment, the method comprises treating an Orthomyxoviridae infection in a mammal in need thereof by administering a therapeutically effective amount of a compound of Formula I or a pharmaceutically acceptable salt, or a composition comprising either. In another aspect of this embodiment; the Orthomyxoviridae infection is an Influenza virus A infection. In another embodiment, the Influenza A virus bears the S31N-mutation. In another aspect of this embodiment, the Orthomyxoviridae infection is an Influenza virus B infection. In another aspect of this embodiment, the Orthomyxoviridae infection is an Influenza virus C infection.

In another embodiment, the method comprises treating an Orthomyxoviridae infection in a mammal in need thereof by administering a therapeutically effective amount of a pharmaceutical composition comprising an effective amount of a Formula I compound, or a pharmaceutically acceptable salt thereof, in combination with at least one additional therapeutic agent. In another aspect of this embodiment, the additional therapeutic agent is a viral haemagglutinin inhibitor, a viral neuramidase inhibitor, a M2 ion channel inhibitor, an Orthomyxoviridae RNA-dependent RNA polymerase inhibitor or a sialidase. In another aspect of this embodiment, the additional therapeutic agent is selected from the group consisting of ribavirin, oseltamivir, zanamivir, laninamivir, peramivir, amantadine, rimantadine, CS-8958, favipiravir, AVI-7100, alpha-1 protease inhibitor and DAS181.

A further aspect of the invention relates to methods of blocking the influx of 14+ ions through the M2-protein ion-channel, inhibiting uncoating and release of free ribonucleoproteins into the cytoplasm, comprising the step of treating with a compound of the invention a sample suspected of containing M2-protein, such as strain A influenza virus, including the S31N strain. Without being bound by a particular theory, compounds of the invention are believed to act by blocking the viral M2-protein functions.

The methods provided herein include administration or use of the compounds, or salts or compositions thereof, described in any of the embodiments or claims set forth herein.

Compounds described herein can be administered as a pharmaceutical composition comprising the compounds of interest in combination with one or more pharmaceutically acceptable carriers. The phrase “therapeutically effective amount” of the present compounds means sufficient amounts of the compounds to treat disorders, at a reasonable benefit/risk ratio applicable to any medical treatment. It is understood, however, that the total daily dosage of the compounds and compositions can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health and prior medical history, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved. Actual dosage levels of active ingredients in the pharmaceutical compositions can be varied so as to obtain an amount of the active compound(s) that is effective to achieve the desired therapeutic response for a particular patient and a particular mode of administration. In the treatment of certain medical conditions, repeated or chronic administration of compounds can be required to achieve the desired therapeutic response. “Repeated or chronic administration” refers to the administration of compounds daily (i.e., every day) or intermittently (i.e., not every day) over a period of days, weeks, months, or longer. Compounds described herein may become more effective upon repeated or chronic administration

Combination therapy includes administration of a single pharmaceutical dosage formulation containing one or more of the compounds described herein and one or more additional pharmaceutical agents, as well as administration of the compounds and each additional pharmaceutical agent, in its own separate pharmaceutical dosage formulation. For example, a compound described herein and one or more additional pharmaceutical agents, can be administered to the patient together, in a single dosage composition having a fixed ratio of each active ingredient; or each agent can be administered in separate dosage formulations. Where separate dosage formulations are used, the present compounds and one or more additional pharmaceutical agents can be administered at essentially the same time (e.g., concurrently) or at separately staggered times (e.g., sequentially).

In one aspect of the invention, compounds of the invention, or a pharmaceutically acceptable salt thereof, or a solvate of either; or (ii) a composition comprising any of the foregoing compound, salt, or solvate and a pharmaceutically acceptable carrier are administered as the active pharmaceutical agent. In another aspect, compounds of the invention or a pharmaceutically acceptable salt thereof, or a solvate of either; or (ii) a composition comprising any of the foregoing compound, salt, or solvate and a pharmaceutically acceptable carrier are administered to a subject and the administered compounds are converted to the active pharmaceutical agent in the subject by chemical or biotransformation.

For oral administration, an effective dose can be expected to be from about 0.0001 to about 100 mg/1 kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight may range from about 3 mg to 1000 mg, about 5 mg to 500 mg, or from about 10 mg to 50 mg, and may take the form of single or multiple doses. For inhaled administration, the daily dose may range from about 1 mg to 200 mg, from about 5 to 100 mg, or from about 10 mg to 50 mg, and may take the form of single or multiple doses.

### Pharmaceutical Compositions

In further embodiments of the invention are pharmaceutical compositions comprising compounds of formula (I), as set forth in the foregoing description, and a pharmaceutically acceptable carrier. Pharmaceutical compositions comprise compounds described herein, pharmaceutically acceptable salts thereof, or solvates of either. The pharmaceutical compositions comprising the compound, salt, or solvate described herein may be formulated together with one or more non-toxic pharmaceutically acceptable carriers, either alone or in combination with one or more other medicaments as described hereinabove.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

The pharmaceutical compositions may be administered orally, rectally, parenterally, intracisternally, intravaginally, intraperitoneally, topically (as by powders, ointments or drops), bucally or as an oral or nasal spray (i.e., inhalation). The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which may serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

For administration by inhalation, the compounds and compositions of the invention may be delivered in an aerosol spray from a pressured container or dispenser, which contains a propellant (e.g., liquid or gas). Administration may be accomplished utilizing a device such as a nebulizer, a metered pump-spray device, dry powder inhaler and a pressurized metered dosing inhaler. A single pressurized metered dose inhaler may be adapted for nasal inhalation routes simply by switching between an actuator that is designed for nasal delivery and an actuator designed for oral delivery. The type of device to deliver compounds and compositions of the invention will depend on the type of targeted inhalation. Useful devices desirably provide consistent measured amounts of aerosolized pharmaceutical compositions thereof for delivery to the oral airway passages and lungs by oral inhalation, or intranasally by inhalation. In certain embodiments, a carrier is used to protect the compounds against rapid elimination from the body, Biodegradable polymers (e.g., ethylene vinyl acetate, polyanhydrides, polyglycolic acid, collagen, polyorthoesters, polylactic acid) are often used. Aerosol formulations typically comprise the active ingredient suspended or dissolved in a suitable aerosol propellant, such as a chlorofluorocarbon (CFC) or a hydrofluorocarbon (HFC). Suitable CFC propellants include trichloromonofluoromethane (propellant 11), dichlorotetrafluoro methane (propellant 114), and dichlorodifluoromethane (propellant 12). Suitable HFC propellants include tetrafluoroethane (HFC-134a) and heptafluoropropane (HFC-227). The propellant typically comprises 40% to 99.5% e.g. 40% to 90% by weight of the total inhalation, composition. The formulation may comprise excipients including co-solvents (e.g. ethanol) and surfactants (e.g. lecithin, sorbitan trioleate and the like). Aerosol formulations are packaged in canisters and a suitable dose is delivered by means of a metering valve (e.g. as supplied by Bespak, Valois or 3M). Methods for the preparation of such formulations are known by those skilled in the art. Powder-based inhalers include reservoir-based devices, containing a bulk container of powder from which several doses may be dispensed, or a supply of unit-doses packaged in blisters, or simple capsules which are loaded by the patient, cut by the device and which deliver the dose of medicinal powder under the suction of patient's inspiratory effort.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. The action of microorganisms may be prevented by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents which delay absorption such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form may be accomplished by dissolving or suspending the drug in an oil vehicle.

Injectable depot forms may be made by forming microencapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations may also prepared by entrapping the drug in liposomes or microemulsions which are compatible with body tissues.

Solid dosage forms for oral administration include capsules, tablets, pills, powders, and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules may be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which may be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, poly(lactic-co-glycolic acid), microcrystalline cellulose, aluminum metahydroxide, bentonite, agar-agar, tragacanth, collagen sponge, demineralized bone matrix, and mixtures thereof.

The compounds may also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes may be used. The present compositions in liposome form may contain, in addition to compounds described herein, stabilizers, preservatives, excipients and the like. The preferred lipids are natural and synthetic phospholipids and phosphatidyl cholines (lecithins) used separately or together. Methods to form liposomes are known in the art. See, for example, Prescott, Ed., Methods in Cell Biology, Volume XIV, Academic Press, New York, N.Y. (1976), p. 33 et seq.

Dosage forms for topical administration of compounds described herein include powders, sprays, ointments and inhalants. The active compounds may be mixed under sterile conditions with a pharmaceutically acceptable carrier and any needed preservatives, buffers or propellants which may be required. Opthalmic formulations, eye ointments, powders and solutions are also contemplated as being within the scope.

The compounds may be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well known in the art. For example, S. M. Berge et al. describe pharmaceutically acceptable salts in detail in (J. Pharmaceutical Sciences, 1977, 66: 1 et seq). The salts may be prepared in situ during the final isolation and purification of the compounds or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts may be prepared in situ during the final isolation and purification of compounds by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol, among others, are equivalent to the unsolvated forms.

### Chemistry

Compounds of the invention may be prepared using a variety of processes well known in the art, such as those set forth in the following schemes. It will be appreciated that the synthetic schemes and specific examples are illustrative and are not to be read as limiting the scope of the invention. Optimum reaction conditions and reaction times for each individual step may vary depending on the particular reactants employed and substituents present in the reactants used. Unless otherwise specified, solvents, temperatures and other reaction conditions may be readily selected by one of ordinary skill in the art. The skilled artisan will also appreciate that not all of the substituents in the compounds of formula (I) will tolerate certain reaction conditions employed to synthesize the compounds. Routine experimentation, including appropriate manipulation of the reaction conditions, reagents and sequence of the synthetic route, protection and deprotection may be required in the case of particular compounds. Suitable protecting groups and the methods for protecting and deprotecting different substituents using such suitable protecting groups are well known to those skilled in the art; examples of which may be found in T. Greene and P. Wuts, Protecting Groups in Chemical Synthesis (3 d ed.), John Wiley & Sons, NY (1999), which is incorporated herein by reference in its entirety.

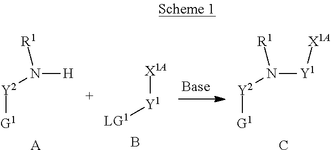

Compounds of formula C, where Y² is a bond or optionally substituted C₁₋₃alkylene, X^{1A} is X¹ or a protected derivative, LG¹ is a leaving group, and G¹, R¹, and Y¹ are as defined herein, may be prepared as generally illustrated in Scheme 1. For example compounds of formula A may be reacted with compounds of formula B in the presence of a suitable base and solvent to provide compounds of formula C. Suitable bases include NaOH, KOH, triethylamine, potassium carbonate and solvents include, for example, water, ethanol, methanol, acetonitrile, tetrahydrofuran, dimethylformamide and the like. Suitable leaving groups LG¹ include chlorides, bromides, iodides, tosylates, mesylates, and the like. Examples of the method of Scheme 1 is shown by the following reactions.

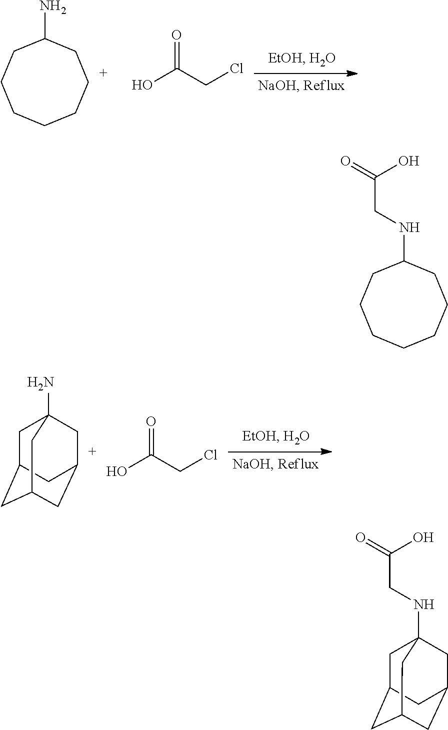

As illustrated by the following reaction, in cases where R¹ is hydrogen, the compound of formula A may be reacted with two equivalents of B to provide formula C, wherein R¹ is Y¹—X^{1A}.

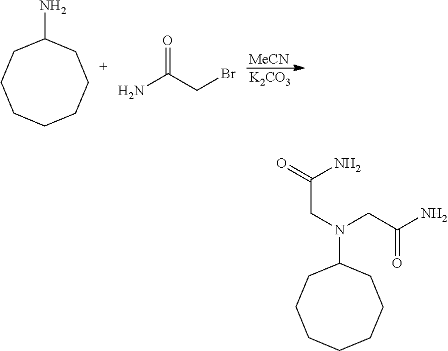

Alternatively, A may be reacted sequentially with two different alkylating agents B to provide compounds where Y¹—X^{1A} are different.

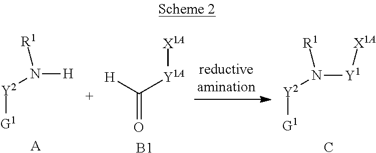

Compounds of formula C may also be prepared by reductive amination as illustrated in Scheme 2, where Y^{lA} is a bond or C₁₋₂alkylene functionalized with an aldehyde. The reductive amination reaction is well known in the art (http://www.organic-chemistry. org/synthesis/C1N/amines/reductiveamination.shtm) and typically involves subjecting the reactants to a borohydride reagent (e.g., NaCNBH₃, NaBH(OAc)₃) in an alcohol solvent like methanol or ethanol. The reaction is illustrated by the following examples.

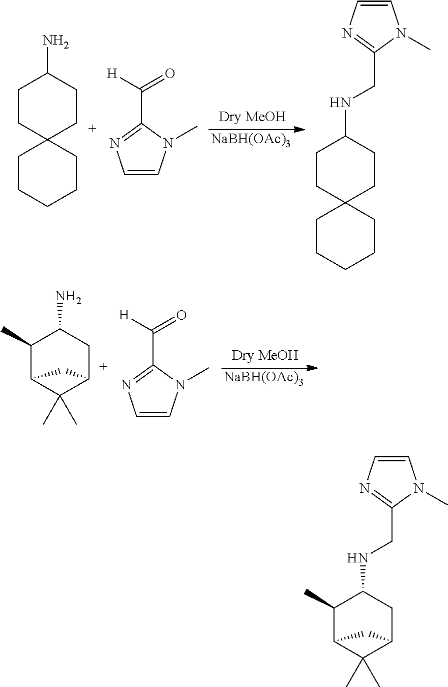

The methods of Schemes 1 and 2 are also applicable to the attachment of Y¹—X^{1A} to the ring nitrogen atom of G², as shown by the following illustration converting A2 to C2.

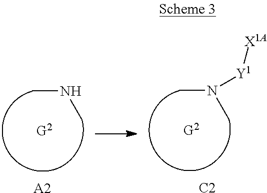

In certain instances, a first X¹ group (or an X^{1A} group) may be transformed into a second X¹ group. Examples include deprotection, alkylating, acylation, oxidation, and reduction reactions that are well known in the art. The following synthetic transformation illustrates an example of transforming a first X¹ group to a second X¹ group by a reduction reaction.

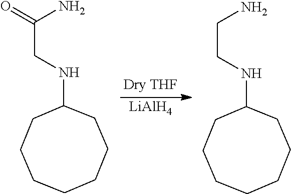

Compounds of formula (I) may be prepared by reacting L¹, and optionally L², with a transition metal salt (e.g., a halide or acetate) in a suitable solvent. In some cases, the reaction may be conducted in the presence of a base to convert the X¹ group to its corresponding salt to facilitate complexation, as in the following examples.

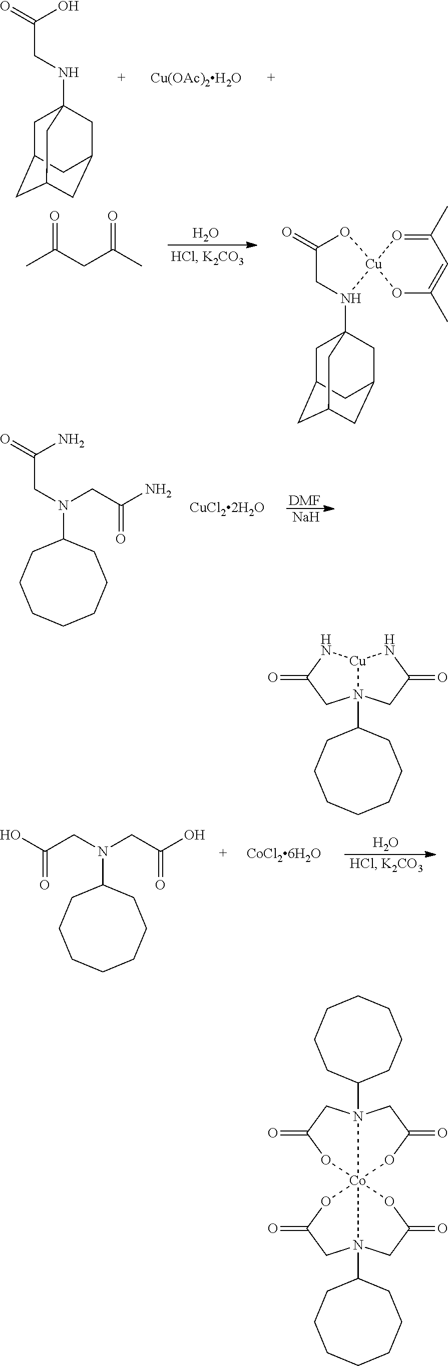

In other cases (e.g., amine functionality in X¹), the base may be omitted:

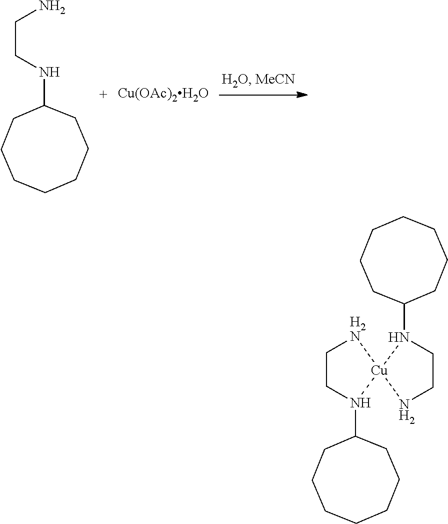

### Examples

¹H and ¹³C NMR spectra were recorded on Varian 300 and 500 MHz multinuclear FT-NMR spectrometers. Proton chemical shifts were reported in parts per million (d) with reference to tetramethylsilane (TMS, δ=0 ppm) or their respective solvent peaks. Mass measurements were done on an Agilent model 61969A LC/MSD TOF mass spectrometer. Melting points were determined using a. Mel-Temp apparatus from Laboratory Devices.

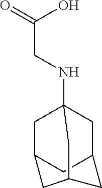 [SMILES: O=C(O)CNC12CC3CC(CC(C3)C1)C2]

### N-(1-adamantyl)-iminoacetic acid (Amt-IMA)

Amantadine HCl (1.88 g, 10 mmol) and chloroacetic acid (1.91 g, 20 mmol) were added to a 50:50 ethanolic aqueous mixture (80 mL) in a 2:1 molar ratio that was then titrated to pH=11.5 using NaOH. The mixture was refluxed at 93° C. for 24 h while maintaining an alkaline pH (11-12). Ethanol was removed from the mixture via rotary evaporation allowing the water insoluble product to precipitate out of solution. Following filtration, the white precipitate was washed with diethyl ether three times (10 mL each) and dried at room temperature. Yield: 1.532 g (73%). ¹HNMR (500 MHz, D₂O, 25° C.): 6=3.40 (s, 2H), 2.02 (s, 3H), 1.72 (s, 6H), 1.48-1.59 (dd, 6H) ppm. ¹³CNMR-¹HNMR (500 MHz, D₂O, 25° C.): 6=172.8, 58.2, 38.9, 36.5, 30.1 ppm. HRMS (ESI): m/z calc for C₁₂H₁₉NO₂+Ft: 210.15. found 210.14. Decomposition point 218° C.

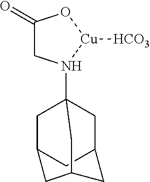

### Copper N-(1-adamantyl)-iminoacetic acid (Amt-IMA-Cu)

Amt-IMA (0.220 g, 1 mmol) and Cu₂CO₃(OH)₂ (0.1183 g, 0.5 mmol) were added to a 50:50 isopropanol/aqueous mixture (100 mL) and were warmed to 55° C. under vacuum for 1 h or until the teal mixture turned light royal blue. Care was taken to not expose the mixture to temperatures above 60° C. or very low pressure to avoid reagent and product loss in the vacuum. Mixing with heat under vacuum was continued for an additional 15 min following the color change of the mixture before cooling to room temperature. The light blue precipitate was filtered from the green-blue solution and dried under vacuum at room temperature. Yield: 0.235 g (58%). ¹HNMR (500 MHz, DMSO-d₆, with trifluoroacetic acid (TFA), 25° C.): 6=3.85 (s, 2H), 2.11 (s, 3H), 1.84 (s, 6H), 1.63 (s, 6H) ppm. HRMS (ESI): m/z calc for [C₁₂H₁₉CuNO₃]⁺: 288.07. found 288.16. Elemental analysis calc (%) for C₁₂H₁₈CuNO₂+3H₂O+HCO₃: C, 40.36; H, 6.51; N, 3.62. found: C, 40.40; H, 5.67; N, 3.64. Decomposed at 204° C.

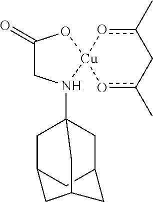

### Copper N-(1-adamantyl)iminoacetate acetylacetonate (Amt-IMA-Cu-Acac)

Procedure 1:

Amt-IMA (0.214 g, 1 mmol) and Cu₂CO₃(OH)₂ (0.1176 g, 0.5 mmol) were added to a 50:50 isopropanol aqueous mixture (100 mL) and warmed to 55° C. under vacuum for 1 h or until the teal mixture turned light royal blue. Mixing with heat under vacuum was continued for an additional 15 min following the color change before adding 2,4-pentanedione (103 μL, 100.4 mg, 1.00 mmol) dropwise and allowing the reaction to ensue under vacuum at 50° C. for 30 min or until the solution turned from blue to a turquoise color. The solution was cooled to room temperature and filtered to remove grayish blue precipitate before precipitating the blue solid product from the transparent 50:50 H₂O:isopropanol solution via slow evaporation under N_{2(g)} at room temperature. Yield: 0.198 g (36%). ¹HNMR (500 MHz, DMSO-d₆, with TFA, 25° C.): 6=8.74-9.79 (d, 1H), 5.65 (s, <1H), 3.83 (s, 2H), 3.66 (s, <1H), 3.14 (s, 4H), 2.11 (d, 4H), 1.99 (s, 2H), 1.81 (s, 6H), 1.53-1.64 (dd, 6H) ppm. HRMS (ESI): m/z calc for [C₁₂H₂₆CuNO₄]⁺: 371.12. found 371.11. Elemental analysis calc (%) for C₁₂H₂₅CuNO₄ (370.11): C, 54.90; H, 7.05; N, 3.77. found: C, 53.71; H, 6.91; N, 3.87. Decomposed at 196° C.

Procedure 2:

CuCl₂.2H₂O (0.0480 g) and Amt-AMA (0.1105 g) were combined in 20 mL of water and 1.0 mL con. HCl while stirring. Powdered K₂CO₃ was added to the stirred solution until the pH reached 6-7; the solution became cloudy. 2,4-pentanedione (0.025 mL) was added to the solution and it was stirred for 30 min. After which, the precipitate was filtered and washed 3 times with 5 mL of water. After air drying overnight, the product weighed 0.1065 g (85% yield if complex has 4 water molecules).

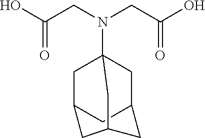 [SMILES: O=C(O)CN(CC(=O)O)C12CC3CC(CC(C3)C1)C2]

### N-(1-adamantyl)-iminodiacetic acid (Amt-IDA)

Amt(9.068 g, 60 mmol) and bromoacetic acid (18.569 g, 134 mmol) were added to a 50:50 ethanolic aqueous mixture (100 mL) in a 2:1 molar ratio that was then titrated to pH=11.5 using NaOH. The mixture was refluxed at 93° C. for 21 h while maintaining an alkaline pH >11. The ethanolic aqueous mixture was extracted with diethyl ether three times (30 mL each) to remove organic impurities. Following extraction and removal of the organic phase, the aqueous phase was titrated to pH 3 with HCl. Slow evaporation in 50:50 ethanol:H₂O afforded the desired white precipitate. Yield: 7.847 g (49%). ¹HNMR (500 MHz, MeOH-d₄, 25° C.): 6=3.89 (s, 4H), 2.21 (s, 3H), 1.97 (s, 6H), 1.73 (t, 6H) ppm. ¹³CNMR-¹HNMR (500 MHz, MeOH-d₄, 25° C.): 6=170.4, 81.4, 65.4, 52.6, 46.9, 35.5, 30.1, 20.5, 12.5 ppm. HRMS (ESI): m/z calc for C₁₄H₂₁NO₂+H⁺: 268.15. found 268.14. Decomposed at 220° C.

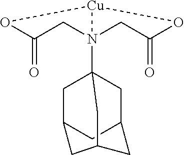

### Copper N-(1-adamantyl)-iminodiacetic acid (Amt-IDA-Cu)

Amt-IDA (0.279 g, 1 mmol) and Cu₂CO₃(OH)₂ (0.119 g, 0.5 mmol) were added to a 50:50 iso-propanol (iPrOH) aqueous mixture (100 mL) in a 2:1 molar ratio and warmed to 55° C. under vacuum for 1 h or until the teal mixture turned light royal blue. Mixing with heat under vacuum was continued for an additional 15 min following the color change of the mixture before cooling at room temperature. The blue solution was filtered under vacuum before crystallizing the blue crystalline product in the residual 50:50 H₂O:iPrOH solution via slow evaporation under N₂(g) at room temperature. The blue crystals were dried in the oven at 80° C. for 3 h. Yield: 0.213 g (43%). ¹HNMR (500 MHz, MeOH-d₄, with TFA, 25° C.): 6=4.19 (s, 4H), 2.24 (s, 3H), 2.01 (s, 6H), 1.73 (t, 6H) ppm. HRMS (ESI): m/z calc for [C₁₄H₂₁CuNO₅]: 346.07. found 346.09. Elemental analysis calc(%) for C₁₄H₂₃CuNO₆ (364.08): C, 46.08; H, 6.35; N, 3.84. found: C, 46.20; H, 6.38; N, 3.73. Decomposed at 180° C.

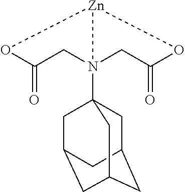

### Zinc adamantyliminodiacetic acid (Amt-IDA-Zn)

Zinc acetate dihydrate (0.1351 g, 0.6155 mmol) was added to a 20 ml of methanol. N-(1-adamantyl)-iminodiacetic acid (Amt-IDA) (0.5032 g, 1.8977 mmol) was added to 2 ml of water. The Amt-IDA solution was dripped into the zinc solution. The solution was heated at reflux for 30 minutes. The solution was evaporated under reduced pressure and left a white solid. The overall yield was 0.0552 g, 27.13%. ¹H NMR (MeOD, 300 MHz) δ=3.68 (d, J=17.2 Hz, 2H), 2.87 (d, J=17.2 Hz, 2H), 2.21 (s, 3H), 1.91 (s, 6H), 1.75 (s, 6H). ¹H NMR (MeOD, 1 drop TFA, 300 MHz) δ ppm: 4.545-3.994 (b, 3H); 3.994-3.796 (s, 1H); 2.441-2.197 (b, 3H); 1.88-1.608 (b, 6H). M/Z (ESI-MS): 210.1522 (Amt-IDA+H). Decomposed at 218° C.

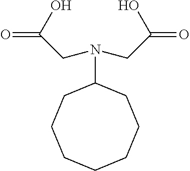 [SMILES: O=C(O)CN(CC(=O)O)C1CCCCCCC1]

### Cylcooctyliminodiacetic acid (CO-IDA

To the reaction flask was added in order: 24 mL ethanol, 6 mL water, 4.0094 g cyclooctylamine (31.513 mmols), and 8.8729 g bromoacetic acid (63.856 mmols). 12M NaOH was added until pH reached 11-12. The pH was monitored until it did not change for an hour. The mixture was refluxed at 94° C. for 24 hours. The solution was cooled to room temperature. An ivory precipitate formed and was collected and rinsed with ethanol 3 times, then dried under vacuum for 24 hours. Mass: 1.9326 g. The remaining solution was filtered 3 times and extracted with diethylether (3×20 ml). A solid formed in the ether layer and was collected and stirred in acetone for 30 minutes, filtered, rinsed 3 times with ethanol, and dried under vacuum for 24 hours. Mass: 2.5156 g. The remaining water ethanol solution was dripped into spinning acetone. A white solid formed and was collected, stirred in ethanol for 20 minutes, filtered, and dried under vacuum for 24 hours. Mass: 3.6547 g. The total yield was 8.1029 g (85%, as the disodium diwater salt). ¹H NMR (D₂O, 300 MHz) δ ppm: 3.218-3.002 (s, 4, CH2), 2.99-2.752 (b, 1, CH), 1.705-1.165 (m, 14, CH2). M/Z (ESI-MS): exact 243.1471. found 244.1548 (M+H). Decomposed at 264° C.

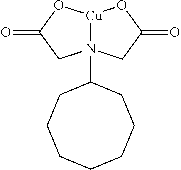

### Copper cyclooctyliminodiacetic acid (CO-IDA-Cu)

Copper acetate monohydrate (0.134 g, 0.6712 mmol) was added to 20 ml of methanol. Cyclooctyliminodiacetic acid (CO-IDA) (0.3984 g, 1.6375 mmol) was added to 2 ml of water. The CO-IDA solution was dripped into the cupric solution, until the hue became a darker blue. The solution was heated at reflux for 30 minutes. The solution was evaporated under reduced pressure and formed a green solid. The overall yield was 0.1222 g (59.77%). ¹H NMR (MeOD, 300 MHz) δ=3.67-1.19 (b). ¹H NMR (MeOD, 1 drop TFA, 300 MHz) δ=4.302-4.004 (s, 4H); 3.988-3.497 (b, 1H); 2.368-1.418 (b, 14H). M/Z (ESI-MS): 244.1555 (CO-IDA+H). Decomposed at 177° C.

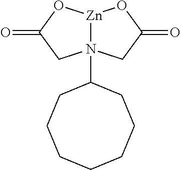

### Zinc cyclooctyliminodiacetic acid (CO-IDA-Zn)

0.1426 g zinc acetate (0.6497 mmols) were added to 20 mL of methanol. 0.3876 g (1.5931 mmols) cyclooctyliminodiacetic acid (CO-IDA) was added to 2 mL of water. The (CO-IDA) solution was dripped into the zinc solution. The solution was heated at reflux for 30 minutes. The solution was rotovaped and formed a solid. The yield was 0.1161 g (58.28%). ¹H NMR (MeOD, 300 MHz) δ=3.92 (s, 2H), 3.40 (m, 1H), 2.11-1.36 (b, 14H). ¹H NMR (MeOD, 1 drop TFA, 300 MHz) δ=4.32-4.029 (s, 4H); 3.853-3.664 (b, 1H); 1.965-1.272 (m, 14). M/Z (ESI-MS):306.0684 (CO-IDA-Zn+H). Decomposed at 220° C.

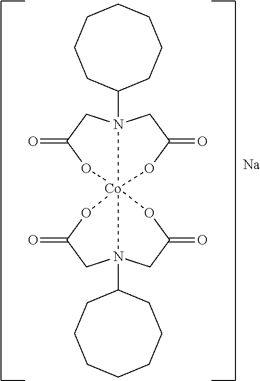

### Cobalt Biscyclooctylimidodiacetic acid (Bis(CO-IDA)-Co)

Cyclooctylimidodiacetic acid (0.0136 g, 0.4260 mmol) was dissolved in 5 ml of water. The solution was treated with 1 M HCl until the pH reached 4. Cobalt(II) chloride hexahydrate (0.0253 g, mmol) was added in 2 ml of water (this gives a 2:1 molar ratio by taking the cyclooctylimidodiacetic acid purity into account). Anhydrous K₂CO₃ was added until the solution reached pH 6. The solution was allowed to sit overnight, and a pink precipitate formed. The precipitate was filtered, washed with 5 ml of water and allowed to air dry. The precipitate weighed 0.0117 g (19.4% yield). ¹H NMR (DMSO, 300 MHz) δ=−12.66-6.00 (b). ¹H NMR (DMSO, 1 drop TFA, 300 MHz) δ=0.85-2.04 (b, 28H), 3.51 (b, 2H), 4.07 (b, 8H). ESI-MS m/z: 244.1554 (CO-IDA+H). Decomposed at 183° C.

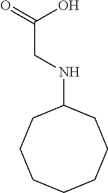 [SMILES: O=C(O)CNC1CCCCCCC1]

### Cyclooctylaminomonoacetic acid (CO-IMA)

Cyclooctylamine (1.552 g, 10 mmol) and 0.9641 g (10 mmol) chloroacetic acid were dissolved in a mixture of 20 ml of methanol and 20 ml of water. 12 M sodium hydroxide was added until the pH reached 11. The mixture was refluxed for 24 hours at 94° C. and maintained at a pH of 11. After letting the reaction cool, the mixture was dripped into 100 ml of stirring acetone. A precipitate formed. The precipitate was collected by vacuum filtration and washed with 10 ml of acetone. The product weighed 0.66 g (21.6% Yield). ¹H NMR (D₂O, 500 MHz) δ=1.23-1.68 (b, 14H), 2.54 (m, 1H), 3.18 (s, 2H). ESI-MS m/z: 186.1552 (M+H). Decomposed at 220° C.

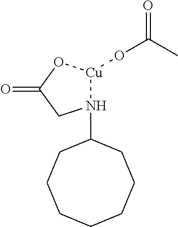

Copper cyclooctylaminoacetic acid (CO-IMA-Cu):

Copper acetate monohydrate (0.1312 g, 0.6572 mmol) was added to a 20 ml of methanol. Cyclooctylaminoacetic acid (CO-IMA) (0.6683 g, 3.5901 mmol) was added to 2 ml of water. The (CO-IMA) solution was dripped into the cupric solution, until the hue became a darker blue. The solution was heated at reflux for 30 minutes. The solution was evaporated under reduced pressure and left a pale blue solid. The yield was 0.1913 g (95.2%). ¹H NMR (MeOD, 1 drop TFA, 500 MHz) δ=1.49-2.51 (b), 2.83-3.1 (b). ¹H NMR (MeOD, 1 drop TFA, 500 MHz) δ=1.98-1.40 (m, 14H), 2.03 (s, 3H), 3.34 (m, 1H), 3.89 (s, 2H). ESI-MS m/z: 186.1536 (CO-IMA+H). Decomposed at 196° C.

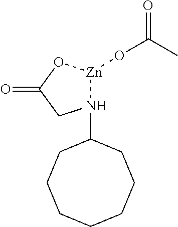

### Zinc cyclooctylaminoacetic acid (CO-IMA-Zn)

Zinc acetate dihydrate (0.1466 g, 0.6679 mmol) was added to a 20 ml of methanol. Cyclooctylaminoacetic acid (0.2975 g, 1.5982 mmol) was added to 2 ml of water. The CO-IMA solution was dripped into the zinc solution. The solution was heated at reflux for 30 minutes. The solution was rotovaped and left a solid. The yield was 0.0845 g (41.12%). ¹H NMR (MeOD, 300 MHz) δ=1.40-1.87 (14H), 3.24 (m, 1H), 3.46 (s, 2H). ¹H NMR (MeOD, 1 drop TFA, 300 MHz) δ=4.284-4.057 (b, 1H); 4.057-3.798 (s, 2H); 2.231-1.426 (m, 14H), 2.03 (s, 3H). M/Z (ESI-MS):186.1536 (CO-IMA+H). Decomposed at 179° C.

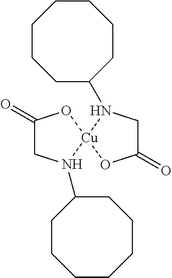

### Copper biscyclooctyl iminomonoacetic acid (Bis(CO-IMA)-Cu)

CO-IMA (0.0714 g, 0.38 mmol) was dissolved in 10 ml of water.

Hydrochloric acid (1 M) was added until the pH reached 3. Copper(II) acetate monohydrate (0.0321 g, 0.16 mmol) was added to the aqueous solution and dissolved by heating and sonication. Anhydrous potassium carbonate was added until the pH reached 7. The solution turned dark blue/purple and a precipitate formed. The solution was allowed to stir for 30 min. The precipitate was collected, washed with 5 ml of water and allowed to dry. The product weighed 0.0231 grams (33% yield). ¹H NMR (MeOD, 500 MHz) δ=1.66-3.62 (b). ¹H NMR (MeOD, drop of TFA, 500 MHz) δ=1.4-2.0 (b, 28H), 3.36 (m, 2H), 3.95 (s, 4H). ESI-MS m/z: 432.2083 (M+H). Decomposed at 178° C. Crystals were obtained by taking a few mg of the product and adding it to 2 ml of water. The container was capped. After 3 weeks, small dark purple crystals began to form on the water-air surface and on the glassware. Verified by single crystal x-ray crystallography.

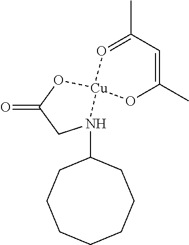

### Copper N-(1-cyclooctyl)iminoacetate acetylacetonate (CO-IMA-Cu-Acac)

CO-IMA (0.0501 g, 0.27 mmol) was added to 10 ml methanol. 1M HCl was added until the pH reached 3 at which point all of the CO-IMA dissolved. Copper(II) acetate monohydrate (0.0540 g, 0.27 mmol) was added to the solution. Water (4 ml) was added to the solution, and the solution was heated and sonicated to dissolve all of the Copper(II) acetate monhydrate. Addition of the copper(II) acetate raised the pH to 6, and the solution turned a cloudy sky blue. Acac (0.0281 g, 28 mmol) was added to the stirring solution. The solution changed color to a deep blue-green and became transparent. The solvent was evaporated by rotary evaporation to yield a solid. The solid was extracted with water, and the extract was filtered. The filtrate was concentrated by rotary evaporation to yield 0.0248 grams of product (26.4% yield). ESI-MS m/z: 347.1130 (M+H).

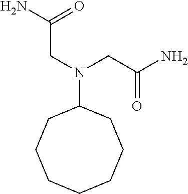 [SMILES: NC(=O)CN(CC(N)=O)C1CCCCCCC1]

### Cyclooctylimidodiacetamide(CO-IDAm)

Cyclooctylamine (0.7715 g, 6 mmol) and 1.6896 g (12.2 mmol) bromoacetamide were dissolved in 20 ml of acetonitrile. Anhydrous K₂CO₃ 1.8455 g (13.3 mmol) was added. The mixture was stirred and heated at 60° C. for 16 hours and allowed to cool. The flask was sonicated to remove any solid adhered to the glass and the precipitate was filtered off; the filtrate was set aside and the precipitate was washed with water. The solid and the filtrate were combined and heated to reflux; the solution became transparent. This solution was allowed to cool and the resulting crystals were collected by vacuum filtration. The product weighed 0.9702 g (66.25% yield). ¹H NMR (DMSO, 300 MHz) δ=1.23-1.80 (b, 14H), 2.58 (m, 1H), 2.92 (s, 4H), 7.17 (s, 2H), 7.72 (s, 2H). ESI-MS m/z: 242.1870 (M+H). Melting point 152-154° C.

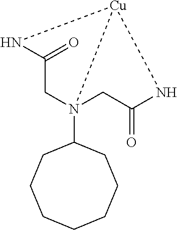

### Copper cyclooctylimidodiacetamide (CO-IDAm-Cu)

CO-IDAm (0.0460 g, 0.1906 mmol) was dissolved in 5 mL dry dimethylformamide. The reaction was placed under nitrogen. NaH (420 mg, 1.750 mmol) was dissolved in 5 mL dry dimethylformamide and added to the CO-IDAm solution. Copper(II) chloride dehydrate (0.0327 g, 0.1918 mmol) dissolved in dry dimethylformamide was slowly dripped into the combined CO-IDAm/NaH solution. The solution started green and gradually changed to dark blue. The reaction was allowed to stir under nitrogen for 16 hours at room temperature. The solvent was evaporated using rotary evaporation. 10 mL of tetrahydrofuran were added to the blue solid and the flask was sonicated. The blue precipitate was filtered and allowed to air dry. The precipitate was extracted with water and the water solution was concentrated to give 0.0231 g of product (Yield 40%). ¹H NMR (MeOD, 500 MHz) δ=1.00-2.50 (b), 2.5-3.7 (b). ¹H NMR (MeOD, 1 drop TFA, 500 MHz) δ=1.20-1.87 (b, 14H), 3.51 (m, 1H), 4.00 (s, 4H). ESI-MS m/z: 242.1870 (CO-IDAm+H). Melting point 142-144° C.

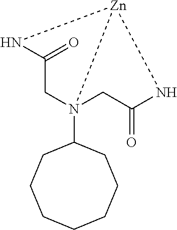

### Zinc cyclooctylimidodiacetamide (CO-IDAm-Zn)

CO-IDAm (0.1056 g, 0.4375 mmol) was dissolved in 5 mL dry dimethylformamide. The reaction was placed under nitrogen. NaH (0.0707 g, 1.747 mmol) were dissolved in 5 mL dry dimethylformamide and added to the CO-IDAm solution. Anhydrous zinc(II) chloride (0.5982 g, 4.388 mmol) dissolved in 5 mL of dry dimethylformamide was slowly dripped into the combined CO-IDAm/NaH solution. The reaction was allowed to stir under nitrogen for 16 hours at room temperature. The solvent was evaporated with a stream of air and gentle heating for 16 hours. The remaining solid was washed with 20 mL of diethyl ether. The solid was washed with 20 mL of methanol. The final product weighed 0.0376 g (Yield 28%). ¹H NMR (DMSO, 300 MHz) δ=1.28-1.92 (b, 14H), 3.13 (b, 1H), 3.40 (b, 4H). ¹H NMR (DMSO, 1 drop TFA, 300 MHz) δ=1.20-1.99 (b, 14H), 3.47 (m, 1H), 3.93 (s, 4H), 7.77 (s, 2H), 7.86 (s, 2H). ESI-MS m/z: 242.1851 (CO-IDAm+H). Decomposed at 244° C.

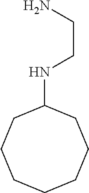 [SMILES: NCCNC1CCCCCCC1]

### Cyclooctylamineethylamine (CO-EA)

Cyclooctylamine (0.2500 g, 1.96 mmol) and 0.2710 g (1.96 mmol) bromoacetamide were dissolved in 15 ml of acetonitrile. Anhydrous K₂CO₃ (0.2717 g, 1.96 mmol) was added. The reaction was stirred and heated at 60° C. for 16 hours. The remaining solid was removed by filtration, and the filtrate was evaporated under vacuum to yield 0.2500 g of the intermediate amide (68.9% yield). The intermediate amide (0.2500 g, 1 mmol) was dissolved in 10 ml dry tetrahydrofuran. LiAlH₄ (0.3276 g, 8.6 mmol) was dissolved in 5 ml tetrahydrofuran. While in an ice bath, the LiAlH₄ solution was slowly added to the monamide solution. The reaction was allowed to stir at 0° C. until the effervescence subsided. The reaction was then heated to 60° C. for 16 hours. The solution was carefully quenched by slow addition of 10 ml of water. The resulting precipitate was filtered and the filtrate was evaporated under vacuum to yield 0.1006 g of a yellow oil. (30% yield). ¹H NMR (D₂O, 500 MHz) δ=1.22-1.60 (b, 14H), 2.44 (t, J=6.2 Hz, 2H), 2.52 (t, J=6.1 Hz, 2H). H—H COSY shows the cyclooctyl ring methine proton under the triplet at 2.52. ESI-MS m/z: 171.1860 (M+H).

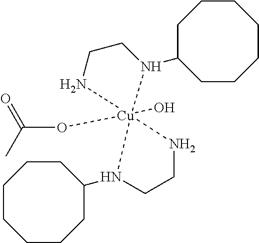

### Copper biscyclooctylethane-1,2-diamine (Bis(CO-EA)-Cu)

Cyclooctylethane-1,2-diamine (0.0347 g, 0.20 mmol) was dissolved in 5 mL water and 10 mL acetonitrile. Copper(II) acetate monohydrate (0.0202 g, 0.10 mmol) in 1 mL of water was dripped into the cyclooctylethane-1,2-diamine solution. The solution turned a dark blue-purple color. The solid was extracted with 5 ml of water. The supernatant was dried by rotary evaporation to yield 0.0362 g (67.7% yield with three waters) of a thick blue oil. ESI-MS m/z: 171.1860 (CO-EA+H). ¹H NMR (D₂O, 300 MHz) δ=1.09-2.03 (b), 3.01-3.52 (b). ¹H NMR (D₂O, 1 drop TFA, 300 MHz) δ=1.16-1.84 (b, 28H), 1.90 (s, 3H), 3.23 (m, 9H). ¹H-¹H COSY shows the methine proton under the multiplet at 3.23 ppm.

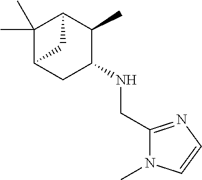 [SMILES: C[C@@H]1[C@H]2C[C@@H](C[C@H]1NCc1nccn1C)C2(C)C]

### Pinanamine imidazole (Pin-Im)

To a solution of 1.00 g (6.5 mmol) (1R,2R,3R,5S)-(−)-isopinocamphenylamine in 25 mL dry methanol, 1.0797 g (9.8 mmol) 1-methyl-2-imidazolecarboxaldehyde were added. It was stirred at room temperature for 1 hour, after which 5.5047 g (26 mmol) triacetoxyborohydride were added. The reaction was then stirred at room temperature for 10 hours. The reaction mixture was quenched by the addition of 30 mL of water. The aqueous layer was extracted with ethyl acetate (3×20 mL). The combined organic layers were washed twice with brine (2×30 mL; brine solution was 7.17 g NaCl in 60 mL deionized water), and then dried using MgSO₄. The solution was then filtered, and the filtrate was dried by rotary evaporation and left on a vacuum to dry overnight. The product was a thick yellow oil. Product weighed 0.4682 g (29% yield). ¹H NMR (CDCl₃, 500 MHz) δ=0.96 (s, 3H), 1.26 (s, 7H), 1.61 (b, 1H), 1.87 (b, 1H), 2.08 (b, 1H), 2.15 (b, 1H), 2.41 (b, 2H), 2.52 (b, 1H), 3.55 (b, 1H), 3.98 (s, 3H), 4.30 (s, 2H), 6.95 (s, 1H), 7.05 (s, 1H). ESI-MS m/z: 248.2179 (M+H).

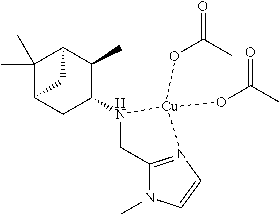

### Copper pinanamine imidazole (Pin-Imid-Cu)

Pinanamine imidazole (0.2696 g, 1.1 mmol) was added to 10 mL of methanol. While the solution was stirring, 0.1186 g (0.59 mmol) copper(II) acetate monohydrate in 20 mL methanol was added (This is a 1:1 molar ratio taking into account the purity of the pinanamine with one arm imidazole). The solution turned a darker blue. No precipitate formed, so the solution was dried by rotary evaporation and then continued to dry overnight on a vacuum to leave a. Product weighed 0.1128 g (55.38% yield). ¹H NMR (CD₃OD, 300 MHz) δ=0.92 (b), 2.66 (b). ¹H NMR (CD₃OD, three drops TFA, 300 MHz) δ=0.99 (s, 3H), 0.99 (m, 1H), 1.22 (b, 6H), 1.63 (b, 2H), 2.08 (s, 6H), 2.12 (b, 1H), 2.30 (b 1H), 2.64 (b, 2H), 2.69 (b, 1H), 3.89 (s, 3H), 4.36 (b, 2H), 7.46 (s, 1H), 7.53 (s, 1H). ESI-MS m/z: 248.2066 (Pin-Im+H).

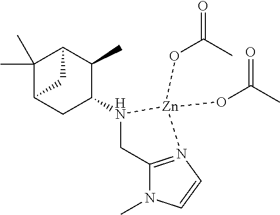

Zinc Pinanamine Imidazole (Pin-Imid-Zn):

Pinanamine imidazole (0.5545 g, 2.2 mmol) was dissolved in 20 mL of methanol. While the solution was stirring, 0.4902 g (2.6 mmol) zinc acetate dihydrate dissolved in 20 mL of methanol was added to the solution (this is approximately a 1:1 molar ratio). The solution was a deep yellow, and no precipitate formed. The solution was then dried by rotary evaporation and then dried on a vacuum overnight. The product weighed 0.4855 g (69.3% yield). ¹H NMR Spectrum 1 (MeOD, 500 MHz) δ=1.02 (s, 3H), 1.17 (m, 1H), 1.28 (b, 6H), 1.90 (m, 2H), 2.02 (m, 1H), 2.22 (m, 1H), 2.41 (m, 1H), 2.55 (m, 1H), 3.39 (m, 1H), 3.76 (s, 3H), 4.08 (d, 1H, J=15.1 Hz), 4.19 (d, 1H, J=15.1 Hz), 7.10 (s, 1H), 7.24 (s, 1H). ¹H NMR Spectrum 2 (MeOD, 1 drop TFA, 500 MHz) δ=1.06 (s, 3H), 1.11 (m, 1H), 1.30 (b, 6H), 2.00 (b, 2H), 2.01 (s, 6H), 2.11 (m, 1H), 2.25 (m, 1H), 2.48 (m 1H), 2.65 (m, 1H), 3.85 (m, 1H), 4.05 (s, 3H), 4.82 (s, 2H), 7.65 (d, J=1.7 1H), 7.67 (d, J=1.7 1H). ESI-MS m/z: 248.2066 (Pin-Im+H).

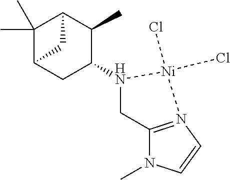

### Nickel pinanamine imidazole (Pin-Imid-Ni)

Pinanamine imidazole (0.0503 g, 0.2 mmol) was dissolved in 10 ml of water. Nickel (II) chloride hexadydrate (0.0483 g, 0.2 mmol) was dissolve in 1 ml of water. The nickel solution was dripped into the pinanamine solution. The solution was allowed to stir overnight and was then evaporated under reduced pressure to give 0.0406 g of product (53.3% yield). ¹H NMR (D₂O, 300 MHz) δ=0.76 (s, 3H), 0.83 (m, 1H), 1.03 (m, 3H), 1.49 (s, 3H), 1.75 (m, 2H), 1.90 (m, 1H), 1.99 (m, 1H), 2.24 (m, 1H), 2.42 (m, 1H), 3.44 (m, 1H), 3.58 (s, 3H), 4.24 (s, 2H), 6.92 (s, 1H), 7.03 (s, 1H). ¹H NMR (D₂O, 1 drop TFA, 300 MHz) δ=0.76 (s, 3H), 0.83 (m, 1H), 1.06 (s, 6H), 1.70 (m, 2H), 2.01 (m 1H), 2.28 (m, 2H), 2.46 (m, 1H), 3.60 (m, 1H), 3.80 (s, 3H), 4.57 (s, 2H), 7.39 (s, 2H). ESI-MS, M/Z. found 248.2121 (Ni-Pin+H).

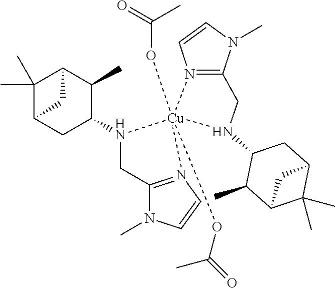

### Copper bispinanamine imidazole (Bis(Pin-Imid)-Cu)

Pinanamine imidazole (0.1179 g, 0.47 mmol) was dissolved in 10 ml of water. Copper(II) acetate monohydrate (0.0460 g, 0.23 mmol) was dissolved in 2 ml of water and was dripped into the stirring pinanamine imidazole solution. The solution turned blue-green. The solution was evaporated under reduced pressure to yield 0.0738 g of product (48.5% yield). ¹H NMR (D₂O, 300 MHz) δ=0.83 (m), 1.02 (d, J=7.0 Hz), 1.09 (s), 1.42 (b), 1.62 (m), 1.76 (m), 1.90 (m), 2.38 (m), 3.47 (m). ¹H NMR (D₂O, 1 drop TFA, 300 MHz) δ=0.80 (s, 6H), 0.84 (m, 2H), 0.94-1.12 (b, 12H), 1.75 (m, 4H), 1.91 (s, 6H), 2.02 (m, 2H), 2.30 (m, 4H), 2.48 (m, 2H), 3.62 (m, 2H), 3.83 (s, 6H), 4.59 (s, 4H), 7.43 (s, 4H). ESI-MS m/z: 248.2127 (Pin-Im+H).

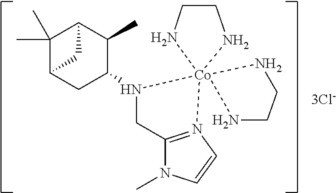

### Cobalt bisethylenediamine pinanamine imidazole (Pin-Imid-Co-Bis(en))

Pinanamine imidazole (0.1662 g, 0.6718 mmol) was dissolved in 15 ml of methanol. Co(en₂Cl₂)Cl was dissolved in 8 ml of methanol and 1 ml of water. The cobalt solution was dripped into the pinanamine solution. The solution was green like the cobalt solution. The solution was refluxed for five minutes and allowed to stir overnight during which time the solution turned a black/pink color. The solvent was removed by rotary evaporation. The blue solid that remained was extracted with ethyl acetate (2×35 ml). The remaining precipitate was collected and washed with 5 ml of ethyl acetate. The final product weighed 0.2295 g. ¹H NMR (CDCl₃, 300 MHz) δ=0.80 (s, 3H), 0.83 (m, 1H), 1.03 (d, J=7.4, 3H), 1.08 (s, 3H), 1.76 (m, 2H), 1.90 (m, 2H), 2.01 (m, 1H), 2.28 (m, 1H), 2.46 (b, 3H), 2.76 (b, 6H), 3.51 (m, 2H), 3.71 (s, 3H), 7.30 (s, 2H). ESI-MS: 248.2108 (Pin-Im+FD.

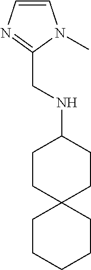 [SMILES: Cn1ccnc1CNC1CCC2(CCCCC2)CC1]

### Spiranamine imidazole (Spi-Imid)

To a solution of 0.5005 g (2.46 mmol) spiranamine in 20 mL dry methanol, 0.4141 g (3.76 mmol) 1-methyl-2-imidazolecarboxaldehyde were added. It was stirred at room temperature for 1 hour, after which 2.7695 g (13 mmol) triacetoxyborohydride were added. The reaction was then stirred at room temperature for 10 hours. The reaction mixture was quenched by the addition of 30 mL of water. The aqueous layer was extracted with dichloromethane (4×20 mL) and the combined organic extracts were dried using MgSO4. The solution was then filtered, and the filtrate was dried by rotary evaporation and left on a vacuum to dry overnight. The product was a thick yellow oil. Product weighed 0.24 g (37% yield). ¹HNMR (CDCl₃, 300 MHz) δ=1.12-2.03 (b, 18H), 3.01 (m, 1H), 3.89 (s, 3H), 4.17 (s, 2H), 6.91 (s, 1H), 7.01 (s, 1H).ESI-MS m/z: 262.2293 (M+H).

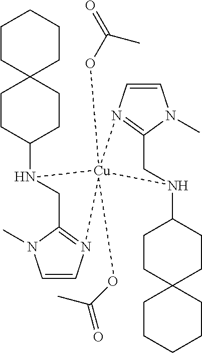

### Copper bisspiranamine imidazole (Bis(Spi-Imid)-Cu)

Spiranamine imidazole (0.2808 g, 1.0 mmol) was dissoloved in 15 ml of methanol. Copper(II) acetate monhydrate (0.2146 g, 1.0 mmol) was dissolved in 30 ml of methanol. The copper solution was dripped into the spiranamine solution with stirring. The solution turned dark blue. The solution was heated to reflux and then cooled. The mixture volume was reduced to half the initial volume with a stream of air. The solution was filtered and the filtrate was concentrated by rotary evaporation. The solid left after rotary evaporation was exctracted with dichloromethane (1×30 ml), and a precipitate remained. The blue dichloromethane solution was evaporated by rotary evaporation. The remaining solid was put on vacuum and weighed 0.2533 g (33% yield). ¹HNMR (DMSO, 500 MHz) δ=0.97-2.25 (b), 2.89 (b). ¹HNMR (DMSO, 1 drop TFA, 500 MHz) δ=0.99-1.78 (b, 36H), 3.17 (b, 2H), 1.88 (s, 6H), 3.87 (s, 6H), 4.56 (b, 4H), 7.91 (b, 2H), 9.36 (b, 2H). ESI-MS m/z: 262.2326 (Spi-Im+H).

### Biological Testing

Part 1. Testing of IMA-Cu and IMA-Cu-ACAC

Liposome Assay

Methods and Materials

Using slight modifications of a previously described protocol, liposomes with or without peptide were prepared in internal buffer (50 mM KCl, 50 mM K₂HPO₄, 50 mM KH₂PO₄, with or without drug, pH 8.0) which was then diluted 100-fold into external buffer (165 mM NaCl, 1.67 mM sodium citrate, 0.33 mM citric acid, with or without drug, pH 6.4) to initiate the experiment with an extra-liposomal pH of 6.3. Liposomes were first prepared by vortexing into internal buffer (1 ml) from a thin film prepared from methanolic E. coli polar lipid (Avanti Polar Lipids, Alabaster, Ala., 20 mg) with or without co-dissolved peptide (M2 22-62, S31N, 0.1 mg, generously provided by Huajun Qin and Timothy A. Cross, synthesized as reported previously′). After three cycles of freezing, thawing, and sonicating, the liposomes were extruded through a filter (200 nm pore size, 21 passages) to produce reasonable uniformity in liposome diameters as described previously. Transport was activated by injection of valinomycin (Sigma-Aldrich, St. Louis, Mo., final concentration: 30 nM, t=60 s), after which residual liposome polarization was relieved with carbonyl cyanide m-chlorophenyl hydrazine (CCCP, Sigma-Aldrich, St. Louis, Mo., final concentration: 1.67 μM, t=120 s). Back-titrations (30 nEq HCl) were then used to calibrate buffer capacity (t=240 and 300 s). Finally, solvent effects of valinomycin and CCCP injections on pH were ascertained with repeat injections (identical volumes) into the depolarized liposome suspension (t=360 and 420 s respectively). The negative controls were blank liposomes (n=4) and liposomes with drug only (n=4 for each concentration); positive controls were liposomes with S31N M2 (22-62) protein only (n=4-8). Effectiveness against proton transport was determined by adding drug to both internal and external buffers at each of three concentrations, 20 μM (n=4), 50 μM (n=4), and 100 μM (n=4), After adjusting for the proton fluxes from the blank and drug only controls, the EC50 values and standard errors were estimated (see below) with the usual single-site blocking function, taking into account the standard error of each sample pool for a given concentration. The control samples were double weighted. These treatments typically perturb the pH by small but measurable amounts (on the order of 0.01 pH units) and are detected with a pH electrode in the external buffer. Proton flux (measured in H+ per tetramer/sec) was calculated from the total initial proton uptake rate increase induced by the valinomycin addition divided by the nominal number of tetramers in the sample calculated using the protein mass determined with UV spectroscopy divided by four times the molecular weight of the monomer (i.e. 4×5,020 Daltons) and multiplied by Avogadro's number.

Results

The table shows the EC₅₀s for the metal compounds against the AMT-resistant proton uptake by proteoliposomes mediated by M2 (22-62, S31N). The percent block was calculating using the EC₅₀s and

$$\% Block = 1 - \frac{100}{1 + \frac{[ x ]}{{EC}_{50}}}$$

and the EC₅₀s were calculated by fitting a sigmoidal binding curve to the three concentration data points using Kaleidagraph.

| Compound | EC₅₀ | % Block |
|---|---|---|
| CuCl₂ | 6.1 | 94% |
| Cu(en)Cl₃ | 20.4 | 83% |
| Amt-IDA-Cu | 21.9 | 82% |
| Amt-IMA-Cu | 18.9 | 84% |
| Amt-IMA-Cu- | 4.5 | 96% |
| ACAC | | |

Miniplaque Assay

Methods and Materials

Cells and media: Tissue used for preparation of virus stock cultures, virus infectivity titrations, and miniplaque drug assays were Madin-Darby Canine Kidney (MDCK) cells (ATCC CRL-2935). The cell culture growth medium used was Dulbecco's Modified Eagles' Medium (DMEM, Sigma-Aldrich) supplemented with 0.11% sodium bicarbonate, 5% Cosmic calf serum (Hyclone), 10 mM HEPES buffer, and 50 μg/ml of penicillin/streptomycin. For culture of virus stocks and virus infectivity assays 0.125% bovine serum albumin (BSA, Sigma-Aldrich) was substituted for the Cosmic calf serum.

Virus: Influenza A virus, the 2009 pandemic strain (A/California/07/2009), was provided by Dr. Brent Johnson, Brigham Young University. Trypsin added to BSA-supplemented media for virus activation was TPCD-treated bovine pancreas trypsin (Sigma-Aldrich). A virus stock culture was prepared in MDCK cells in a 150 cm² culture flask. The cells were planted in growth medium and incubated until the cell monolayer was at 90% confluency. The monolayer was washed with medium containing no serum (serumless medium), then renewed with BSA medium containing 2.5 μg/ml of trypsin. The culture was infected with 1 ml of the virus inoculum obtained from Dr. Johnson, then incubated at 33° C. At 16 hours post-infection the culture is decanted. Culture is fixed in 1 mL cold acetone and allowed to sit for 10-15 min. The coverslips were then removed and allowed to air dry for 30 minutes at room temperature. Coverslips were subsequently died with 23 μl of antibody reagent which was distributed evenly over the area of the coverslip. The coverslips were then incubated in a humidified chamber at 37° C. for 30 minutes. After incubation, coverslips were gently washed in a stream of PBS-Tween, and distilled water. Excess fluid was removed by touching the side of the coverslip on a Kimwipe and mounting cell side down on a small drop of mounting fluid cell side down. Specimen was then observed under a microscope.

Procedure. In cell culture, mini-plaques consist of single infected cells, double or multiple infected cells contiguously linked, that are observed microscopically and identified by immunofluorescence using FITC-labeled monoclonal antibody against viral protein. Antiviral activity of test drugs were detected in cultures exposed to drug by assessing inhibition of viral protein synthesis as measured by reduction in number of mini-plaques. The tests were performed in MDCK cells. Cells were grown on 12-mm glass coverslips in shell vials (Sarstadt) to a cell density of 80%-99% confluency in 1 ml of DMEM growth medium per vial. Prior to infection the cultures were washed with serumless media. The serumless medium was replaced with 1 ml per vial of DMEM containing BSA at a concentration of 0.125%. Test drugs at concentrations of 50 μM were added to the cultures and allowed to equilibrate with the media. Stock virus was thawed and appropriate concentrations of virus (contained in BSA media) were then exposed to 1.0 μg/ml of trypsin for 30 minutes at room temperature, then added to the cultures. Replicate cultures were included at each dilution step of test chemical. Control cultures containing no antiviral drug were included in each assay. The cultures were then incubated at 33° C. for 16 hours. Cultures were washed with phosphate buffered saline (PBS) within the shell vials, fixed in −80° C. acetone, then stained with anti-Influenza A, FITC-labeled monoclonal antibody (Millipore, Billerica, Mass., USA). Possible drug toxicity in culture was assessed by microscopic observation of cytologic changes and cell multiplication rates.

EC₅₀ determinations were carried out with a fluorescence microscope by counting miniplaques (clusters of infected cells, typically 80-100 per cover slip in control samples and fewer in cultures treated with active drugs) in a confluent MDCK monolayer on a cover slip at drug concentrations of 50 μM. The following equation for miniplaque count was fitted to the data, where D is drug concentration and C₀ is the miniplaque count in drug-free controls.

$$C ( D ) = \frac{{C}_{0}}{1 + \frac{D}{{EC}_{50}}}$$

Results

The table below shows the effect of several synthesized complexes on the infectivity of influenza A (S31N) in MDCK cells. MDCK cells were infected in the presence or absence of test compounds. The number of miniplaques formed correlates to the effectiveness of test compounds. The percent block for each compound was calculated by comparing the average number of plaques for a given test compound to the average number of plaques for the coverslips without any test compounds. The EC50 was then calculated from the percent block data for each compound.

| Compound | EC₅₀ | % Block |
|---|---|---|
| CuCl₂ | 57.2 | 63% |
| Cu(en)Cl₂ | 8.7 | 92% |
| Cu(en)₂Cl₂ | 45.2 | 69% |
| Cu(dien)Cl₂ | 51.6 | 66% |
| Amt-IDA-Cu | 21.8 | 82% |
| Amt-IMA-Cu | 25.7 | 80% |
| Amt-IMA-Cu | 2.91 | 97% |
| ACAC | | |

Part 2. Testing of Additional Compounds

Oocyte Assay

Methods and Materials

Microinjection and Culture of Oocytes: Xenopus laevis oocytes from Ecocyte (Austin, Tex.) were maintained in ND-96++ solution (96 mM NaCl, 2 mM KCl, 1.8 mM CaCl2.1 mM MgCl2, 2.5 mM pyruvic acid, 5 mM HEPES, pH 7.4) after injection of 100 ng/mL of mRNA within one day of shipping.

Electrophysiological Recordings: 72-96 h after mRNA injection, whole-cell currents were recorded with a two-electrode voltage-clamp apparatus (Axon Instruments DIGIDATA 1322A) that recorded the voltage difference between a pipette (filled with 3 M KCl) located in the cell and another in the surrounding bath. A voltage-clamp amplifier (Axon Instruments GeneClamp 500B) provided feedback current to the oocyte through a second intracellular pipette. Oocyte currents were recorded in standard Barth's solution (0.3 mM NaNO3, 0.71 mM CaCl2, 0.82 mM MgSO4, 1.0 mM KCl, 2.4 mM NaHCO3, 88 mM NaCl, 15.0 mM HEPES, pH 7.4) or Barth's solution titrated with HCl to pH 5.3. The metal and non-metal complexes were diluted in the Barth's (pH 5.3) from 10 mM to 100 μM. To check that the oocytes did not develop non-specific leakage currents during the recordings, we applied standard Barth's solution (pH 7.4) for 2 min at the end of the measurements from each oocyte.

Results: The table below shows the effect of the compounds on currents through influenza A M2 channels (A/Udorn/72 strain background but with the amantadine-insensitive S31N mutant) found in transfected Xenopus laevis oocytes. In each experiment, the perfusion with 4 μM and then 20 μM lasted 1 minute, i.e. long enough to ascertain whether the drug was a rapid, very strong blocker. In no case was this true. However, the subsequent perfusion with 100 μM drug allowed us to determine whether the drugs have efficacy at the same level as amantadine in amantadine-sensitive type (S31) M2 channels. Where the Percent Block exceeds 50% and the Percent Washout is less than 50%, this represents approximate therapeutic level judging from the history of amantadine usage in infected humans. The EC₅₀ is an estimated “50% Effect” concentration obtained using the percentage blocks after 1 minute in the three concentrations. For the copper compounds, this is a conservative underestimate because the perfusions were not long enough to allow complete block equilibration.

No Metal Udorn 72 S31N

| | | Percent | Percent | |
|---|---|---|---|---|
| Compound | EC₅₀^{a} | Block^{b} | Washout^{c} | |
| CO-IDAm | 130 μM | 17% | 100% | N = 2 |
| CO-IDA | N/A | No Block | N/A | N = 2 |
| Pin-Imid | N/A | No Block | N/A | N = 2 |

With Metal Udorn 72 S31N

| | | | Percent | Percent | |
|---|---|---|---|---|---|
| Compound | EC₅₀ | | Block | Washout | |
| Cu(Acetate)2 | 74 | μM | 70% | 4% | N = 2 |
| CoCl₂ | >>100 | μM | 1% | 100% | N = 2 |
| Bis(CO-IMA)-Cu | 41 | μM | 81% | 11% | N = 2 |
| Bis(Pin-Imid)-Cu | 115 | μM | 45% | 8% | N = 2 |
| Bis(CO-EA)-Cu | 71 | μM | 67% | 16% | N = 2 |
| CO-IDAm-Cu | 37 | μM | 86% | 10% | N = 2 |
| CO-IDA-Cu | 164 | μM | 26% | 94% | N = 2 |
| CO-IMA-Cu-ACAC | 64 | μM | 73% | 16% | N = 2 |
| Amt-IMA-Cu-ACAC | 57 | μM | 65% | 58% | N = 2 |
| Pin-Imid-Cu | 39 | μM | 83% | 66% | N = 2 |
| CO-IDA-Zn | 208 | μM | 32% | 80% | N = 2 |
| CO-IMA-Zn | 125 | μM | 42% | 93% | N = 2 |
| Pin-Imid-Zn | 113 | μM | 39% | 22% | N = 2 |
| CO-IDAm-Zn | 137 | μM | 42% | 74% | N = 2 |
| Bis(CO-IDA)-Co | 240 | μM | 28% | 89% | N = 2 |

| | | | | |
|---|---|---|---|---|
| Pin-Imid-Co-Bis(en) | N/A | No Block | N/A | N = 2 |

Compounds complexed with and without metal tested using the two-electrode voltage clamp and Xenopus laevis oocytes at three concentrations per oocyte (4 μM, 20 μM, and 100 μM). The EC₅₀ was calculated using Kaleidagraph by calculating the percent activity at 4, 20, and 100 μM and fitting a sigmoidal binding curve to the percent activity data points. The percent block was calculated as 1-(inward current remaining after 1 min at 100 μM/inward current with no compound). The percent washout was calculated using (remaining inward current after 1 min at 100 μM inward current after 1 min of washout)/(remaining inward current at 100 μM inward current with no compound).

Liposome Assay

Methods

Liposome Preparation

Test-tubes were sterilized by washing with ethanol, acetone, chloroform and then petroleum ether. The test tubes were then air dried upside down. E. coli lipid extract in chloroform was added to each test-tube, and the solution was then rotovaped under nitrogen until all the chloroform was evaporated and a thin film of lipids had formed at the bottom of the test-tube. After covering the test tubes with Parafilm to reduce oxidation of the lipids, M2 22-62 peptide (either “wild type” A/Udorn/72 or “mutant” A/Udorn/72 S31N) in methanol was added to the thin film with equal amounts of chloroform. This mixture was vortexed and sonicated until the film was in solution, and the resulting solution was then dried under nitrogen gas. Warmed internal buffer (a solution containing 50 mM each of KCl, KH2PO4, and K2HPO4) was added, and the mixture was again vortexed and sonicated. At this point, the liposomes were extruded (passed through a 200 nm filter) to ensure a small, uniform size. The extruder components and syringes were cleaned with ethanol, heated in an incubator to 50-55° C., assembled, and then rinsed through with internal buffer 11 times before the liposome solution was pushed through 21 times. The liposomes were collected in Eppendorf tubes and incubated at room temperature for 24 hours before use.

Assay Procedure

3 mL of external buffer (a solution containing 165 mM NaCl, 1.67 mM Na+ citrate, and 0.33 mM citric acid) followed by 30μL of 0.1 M HCl were added to a shell vial. While stirring, 30μL of drug was added, followed by 30μL of liposomes. A pH electrode was inserted into the cuvette. During the course of assay, the solution inside the cuvette was stirred constantly. Throughout the assay, injections were made at 0.25 s intervals to determine the proton flux through the M2 ion channel. The time sequence of the procedure was: 70 s after beginning the assay, 4μL of 25 mg/mL valinomycin (in ethanol) was injected; 130 s: 25μL of 200μM CCCP (in ethanol) was injected; 250 s and 310 s: 30μL of 0.001 M HCl was added; 370 s: a second valinomycin injection was made; 450 s: a second CCCP injection was made. The final two injections were made after all liposomes were depolarized and demonstrated the direct effects of the chemicals on the buffer pH. This information was used to gauge which portion of the original signals was due to valinomycin-induced proton uptake and the total initial polarization level. Some experiments with weak initial polarization due to contamination or weak positive protein control induced proton uptake due to protein measurement errors were excluded.

The assay was carried out both with protein-containing and protein-free liposomes. The protein-free “blanks” were prepared in the same way, except without adding M2 protein. The blanks without drug were used to evaluate the integrity of the lipid, and the blanks with drug to evaluate the bilayer-permeabilizing effect of the drug.

| | Wild Type | | | Wild Type w/ | | | Wild Type |
|---|---|---|---|---|---|---|---|
| | Proton | Standard | | Compound Proton | Standard | | Percent |
| Compounds | Flux^{a} | Deviation | N = | Flux^{a} | Deviation | N = | Block^{b} |
| CO-IDA-Cu | 28.328742 | 6.676771 | 2 | 16.18902459 | 1.11902459 | 3 | 48.72% |
| CO-IDA-Zn | 34.1895225 | 1.37810877 | 2 | 15.8914833 | 4.516486 | 2 | 66.00% |
| Bis-CO- | 24.9246074 | 3.36160582 | 2 | 12.0377672 | 2.10003005 | 2 | 71.43% |
| IMA-Cu | | | | | | | |
| Cu(AC)2 | 20.4465979 | 5.39587521 | 3 | 10.2193317 | 0.93045975 | 2 | 63.19% |
| | Mutant | | | Mutant w/ | | | Mutant |
| | Proton | Standard | | Compound Proton | Standard | | Percent |
| Compounds | Flux^{a} | Deviation | N = | Flux^{a} | Deviation | N = | Block^{c} |
| CO-IDA-Cu | 44.6977972 | 12.3982598 | 2 | 12.8894733 | 3.58067056 | 3 | 75.75% |
| CO-IDA-Zn | 36.77867 | 3.53297779 | 3 | 13.2113132 | 1.64754493 | 3 | 76.29% |
| Bis-CO- | 40.7359631 | 16.8089914 | 2 | 16.9518655 | 0.66350543 | 3 | 70.38% |
| IMA-Cu | | | | | | | |
| Cu(AC)2 | 45.8308727 | 17.4265191 | 2 | 17.8461993 | 0.5073256 | 3 | 67.23% |

^{a}H⁺/sec/channel. The valinomycin initial slope (after the artifact) is divided by the average value of the back-titrations, which is then divided by half the nominal number of tetramers (because 50% of the channels are found to be oriented backwards in the liposomes and presumed to be non-functional due to the alkaline liposome interior).
^{b}{1 − (Wild-type w/Compound flux − Blank w/Compound Flux)]/(Wild-type Flux − Blank Flux)} × 100% = % Block of wild type. (Blank fluxes not shown).
^{c}{1 − (Mutant w/Compound flux − Blank w/Compound Flux)/(Mutant Flux − Blank Flux)} × 100% = % Block. (Blank fluxes not shown).

Miniplaque Assay

Methods

Cells and media: Tissue used for preparation of virus stock cultures, virus infectivity titrations, and miniplaque drug assays were Madin-Darby Canine Kidney (MDCK) cells (ATCC CRL-2935). The cell culture growth medium used was Dulbecco's Modified Eagles' Medium (DMEM, Sigma-Aldrich) supplemented with 0.11% sodium bicarbonate, 5% Cosmic calf serum (Hyclone), 10 mM HEPES buffer, and 50 μg/ml of penicillin/streptomycin. For culture of virus stocks and virus infectivity assays 0.125% bovine serum albumin (BSA, Sigma-Aldrich) was substituted for the Cosmic calf serum.

Virus: Influenza A virus, the 2009 pandemic strain (A/California/07/2009), was provided by Dr. Brent Johnson, Brigham Young University. Trypsin added to BSA-supplemented media for virus activation was TPCD-treated bovine pancreas trypsin (Sigma-Aldrich). A virus stock culture was prepared in MDCK cells in a 150 cm² culture flask. The cells were planted in growth medium and incubated until the cell monolayer was at 90% confluency. The monolayer was washed with medium containing no serum (serumless medium), then renewed with BSA medium containing 2.5 μg/ml of trypsin. The culture was infected with 1 ml of the virus inoculum obtained from Dr. Johnson, then incubated at 33° C. At 16 hours post-infection the culture is decanted. Culture is fixed in 1 mL cold acetone and allowed to sit for 10-15 min. The coverslips were then removed and allowed to air dry for 30 minutes at room temperature. Coverslips were subsequently died with 23 μl of antibody reagent which was distributed evenly over the area of the coverslip. The coverslips were then incubated in a humidified chamber at 37° C. for 30 minutes. After incubation, coverslips were gently washed in a stream of PBS-Tween, and distilled water. Excess fluid was removed by touching the side of the coverslip on a Kimwipe and mounting cell side down on a small drop of mounting fluid cell side down. Specimen was then observed under a microscope.

Procedure. In cell culture, mini-plaques consist of single infected cells, double or multiple infected cells contiguously linked, that are observed microscopically and identified by immunofluorescence using FITC-labeled monoclonal antibody against viral protein. Antiviral activity of test drugs were detected in cultures exposed to drug by assessing inhibition of viral protein synthesis as measured by reduction in number of mini-plaques. The tests were performed in MDCK cells. Cells were grown on 12-mm glass coverslips in shell vials (Sarstadt) to a cell density of 80%-99% confluency in 1 ml of DMEM growth medium per vial. Prior to infection the cultures were washed with serumless media. The serumless medium was replaced with 1 ml per vial of DMEM containing BSA at a concentration of 0.125%. Test drugs at concentrations of 50 μM were added to the cultures and allowed to equilibrate with the media. Stock virus was thawed and appropriate concentrations of virus (contained in BSA media) were then exposed to 1.0 μg/ml of trypsin for 30 minutes at room temperature, then added to the cultures. Replicate cultures were included at each dilution step of test chemical. Control cultures containing no antiviral drug were included in each assay. The cultures were then incubated at 33° C. for 16 hours. Cultures were washed with phosphate buffered saline (PBS) within the shell vials, fixed in −80° C. acetone, then stained with anti-Influenza A, FITC-labeled monoclonal antibody (Millipore, Billerica, Mass., USA). Possible drug toxicity in culture was assessed by microscopic observation of cytologic changes and cell multiplication rates.

EC₅₀ determinations were carried out with a fluorescence microscope by counting miniplaques (clusters of infected cells, typically 80-100 per cover slip in control samples and fewer in cultures treated with active drugs) in a confluent MDCK monolayer on a cover slip at drug concentrations of 50 μM. The following equation for miniplaque count was fitted to the data, where D is drug concentration and C₀ is the miniplaque count in drug-free controls.

$$C ( D ) = \frac{{C}_{0}}{1 + \frac{D}{{EC}_{50}}}$$

Results

The table below shows the effect of several synthesized complexes on the infectivity of influenza A (S31N) in MDCK cells. MDCK cells were infected in the presence or absence of test compounds. The number of miniplaques formed correlates to the effectiveness of test compounds. The percent block for each compound was calculated by comparing the average number of plaques for a given test compound to the average number of plaques for the coverslips without any test compounds. The EC50 was then calculated from the percent block data for each compound.

| Compound | % Block | EC₅₀ (μM) |
|---|---|---|
| CO-IDA-Cu | 62.2 | 30.3 |
| Pin-Imid-Cu | 71.6 | 19.9 |
| Bis(CO-IMA)-Cu | 66.4 | 25.3 |
| CO-IDAm-Cu | 60.2 | 33.5 |
| Bis(CO-EA)-Cu | 94.5 | 2.91 |
| CO-IMA-Cu | 80.1 | 6.2 |
| Cu(Acetate)₂ | 38 | 40.8 |
| CO-IMA-Cu-ACAC | 65.7 | 13.1 |
| Cyclen-Cu | 38.7 | 39.6 |
| CO-IMA-Zn | 79.8 | 12.6 |
| CO-IDA-Zn | 61.5 | 31.3 |
| Co-IDAm-Zn | 54 | 21.3 |
| Bis(CO-IDA)-Co | 50.6 | 24.4 |
| Pin-Imid-Co-Bis(en) | 65.6 | 13.1 |
| CoCl₂ | 61 | 16 |

The above description of the examples and embodiments of the invention is merely exemplary in nature and, thus, variations thereof are not to be regarded as a departure from the spirit and scope of the invention.

1. A pharmaceutical composition comprising a compound of formula (I), or a salt thereof, and a pharmaceutically acceptable carrier
(L¹)ₘM^{p+}(L²)ₙ (I)
wherein:
M is a transition metal, where p is an integer of from 0 to 5;
m is 1, 2 or 3;
each L¹ is independently
a) G¹-Y²—N(R¹)—Y¹—X¹,
b) G¹-Y²—N(—Y¹—X¹)₂, or
c) G²(-Y¹—X¹)ᵣ;
each R¹ is independently H or C₁₋₆alkyl;
each X¹ is independently OH, OC₁₋₄alkyl, NH₂, NH(C₁₋₄alkyl), N(C₁₋₄alkyl)(C₁₋₄alkyl), COOH, CONH₂, CONH(C₁₋₄alkyl), CON(C₁₋₄alkyl)(C₁₋₄alkyl), C(NH)NH₂, NHC(NH)NH₂, NHOH, SH, S(C₁₋₄alkyl), C(NC₁₋₄alkyl), a 5- or 6-membered nitrogen-containing heteroaryl, or a 4- to 8-membered nitrogen-containing heterocycle, or salts thereof, the 5- or 6-membered nitrogen-containing heteroaryl and the 4- to 8-membered nitrogen-containing heterocycle each being independently optionally substituted with 1-4 substituents independently selected from the group consisting of C₁₋₄alkyl, C₁₋₄haloalkyl, halo, C₁₋₄alkoxy, and C₁₋₄haloalkoxy;
each Y¹ is independently C₁₋₃alkylene or a bond;
each Y² is independently a bond or C₁₋₃alkylene, the C₁₋₃alkylene being optionally substituted with hydroxy, NH₂, NH(C₁₋₄alkyl), or N(C₁₋₄alkyl)(C₁₋₄alkyl);
G¹ is
a) an alicyclyl, the alicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;
b) a heteroalicyclyl, the heteroalicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;
c) a silacyclyl, the silacyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkyl, halo, C₁₋₆alkoxy, and C₁₋₆haloalkoxy;
d) C₆₋₂₀aryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy; or
e) a 5- to 20-membered heteroaryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
G² is
a) a heteroalicyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy; or
b) a silacyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl C₁₋₆haloalkyl, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
r is 1 or 2;
each L² is independently an auxiliary ligand, L² being a monodentate, bidentate, tridentate, or tetradentate ligand;
and
n is an integer from 0 to 4.

2. A compound of formula (I), or a salt thereof,
(L¹)ₘM^{p+}(L²)ₙ (I)
wherein:
M is a transition metal, where p is an integer of from 0 to 5;
m is 1, 2 or 3;
each L¹ is independently
a) G¹-Y²—N(R¹)—Y¹—X¹,
b) G¹-Y²—N(—Y¹—X¹)₂, or
c) G²(-Y¹—X¹)ᵣ;
each R¹ is independently H or C₁₋₆alkyl;
each X¹ is independently OH, OC₁₋₄alkyl, NH₂, NH(C₁₋₄alkyl), N(C₁₋₄alkyl)(C₁₋₄alkyl), COOH, CONH₂, CONH(C₁₋₄alkyl), CON(C₁₋₄alkyl)(C₁₋₄alkyl), C(NH)NH₂, NHC(NH)NH₂, NHOH, SH, S(C₁₋₄alkyl), C(NC₁₋₄alkyl), a 5- or 6-membered nitrogen-containing heteroaryl, or a 4- to 8-membered nitrogen-containing heterocycle, or salts thereof, the 5- or 6-membered nitrogen-containing heteroaryl and the 4- to 8-membered nitrogen-containing heterocycle each being independently optionally substituted with 1-4 substituents independently selected from the group consisting of C₁₋₄alkyl, C₁₋₄haloalkoxy, halo, C₁₋₄alkoxy, and C₁₋₄haloalkoxy;
each Y¹ is independently C₁₋₃alkylene or a bond;
each Y² is independently a bond or C₁₋₃alkylene, the C₁₋₃alkylene being optionally substituted with hydroxy, NH₂, NH(C₁₋₄alkyl), or N(C₁₋₄alkyl)(C₁₋₄alkyl);
G¹ is
a) an alicyclyl, the alicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
b) a heteroalicyclyl, the heteroalicyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
c) a silacyclyl, the silacyclyl being optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
d) C₆₋₂₀aryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy; or
e) a 5- to 20-membered heteroaryl optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
G² is
a) a heteroalicyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy; or
b) a silacyclyl having one nitrogen as a ring atom and optionally substituted with 1-6 substituents independently selected from the group consisting of hydroxy, oxo, NH₂, NH(C₁₋₄alkyl), N(C₁₋₄alkyl)(C₁₋₄alkyl), C₁₋₁₀alkyl, C₁₋₆haloalkoxy, C₃₋₁₂alicyclyl, 4- to 8-membered heterocyclyl, C₆₋₁₂aryl, halo, C₁₋₆alkoxy, and C₁₋₆haloalkoxy, the C₃₋₁₂alicyclyl, 4- to 8-membered heterocyclyl, and C₆₋₁₂aryl being optionally substituted with 1-4 substituents independently selected from hydroxy, oxo, NH₂, NH(C₁₋₄alkyl), N(C₁₋₄alkyl)(C₁₋₄alkyl), C₁₋₁₀alkyl, C₁₋₆haloalkoxy, halo, C₁₋₆alkoxy, and C₁₋₆haloalkoxy;
r is 1 or 2;
each L² is independently an auxiliary ligand, L² being a monodentate, bidentate, tridentate, or tetradentate ligand;
and
n is an integer from 0 to 4;
with the proviso that the compound of formula (I) excludes:
diaqua(N-(1-adamantyl)-iminodiacetate)copper(II);
bis-[(imidazole)(N-(1-adamantyl)-iminodiacetate)copper(II)];
(2,2′-bipyridine)(N-(1-adamantyl)-iminodiacetate)copper(II);
((1S,2S,3S,5R)-2,6,6-trimethyl-N-((1-methyl-1H-imidazol-2-yl)methyl)-bicyclo[3.1.1]heptan-3-amine)copper(II)diacetate or hydrate or solvate thereof; and
((1 S,2S,3S,5R)-2,6,6-trimethyl-N-((1-methyl-1H-imidazol-2-yl)methyl)-bicyclo[3.1.1]heptan-3-amine)copper(II)dichloride or hydrate or solvate thereof.

3. A method of treating influenza A comprising administering to a patient in need thereof, a therapeutically effective amount of the composition of claim 1 or salt thereof.

4. The composition of claim 1 or salt thereof, wherein M is selected from the group consisting of Cu, Zn, Ni, Co, Fe, Mn, Cr, V, Ti, Ag, Pd, Rh, Ru, Mo, Au, Pt, Ir, and W.

5-6. (canceled)

7. The composition of claim 1 or salt thereof, wherein G¹ is selected from the group consisting of a monocyclic cycloalkyl, a monocyclic cycloalkenyl, a bicyclic cycloalkyl, a bicyclic cycloalkenyl, or a tricyclic cycloalkyl, the monocyclic cycloalkyl, the monocyclic cycloalkenyl, the bicyclic cycloalkyl, the bicyclic cycloalkenyl, and the tricyclic cycloalkyl being optionally joined to a second alicyclic ring to form a spirocyclic ring system, G¹ being optionally substituted as defined in claim 1.

8. (canceled)

9. The composition of claim 1 or salt thereof, wherein each Y¹—X¹ is independently selected from the group consisting of

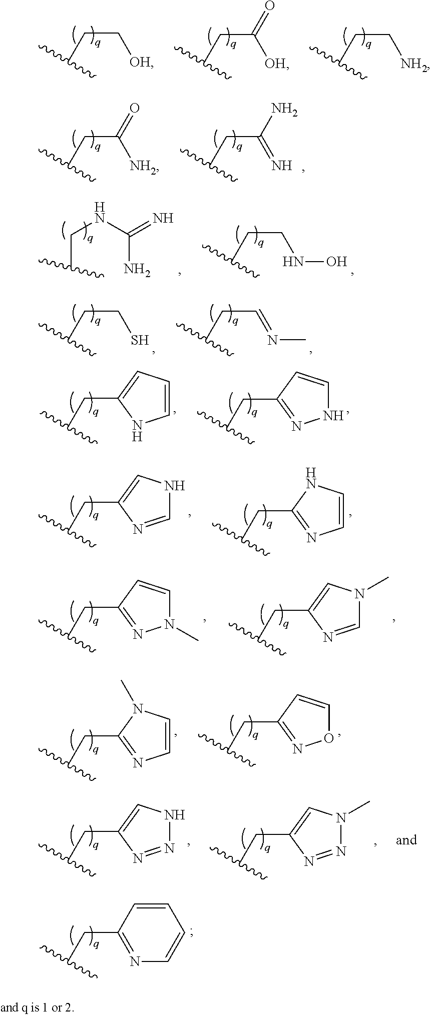

10. The composition of claim 1 or salt thereof, wherein each X¹ is independently NH₂, COOH, CONH₂, 1-methyl-1H-imidazol-2-yl, or salts thereof.

11. The composition of claim 1 or salt thereof, wherein Y¹—X¹ is selected from the group consisting of —CH₂CH₂NH₂, —CH₂COOH, —CH₂CONH₂, and (1-methyl-1H-imidazol-2-yl)methyl, or salts thereof.

12-15. (canceled)

16. The composition of claim 1 or salt thereof, wherein each L¹ is independently G¹-Y²—N(R¹)—Y¹—X¹, and G¹-Y²—N(R¹)— is selected from:

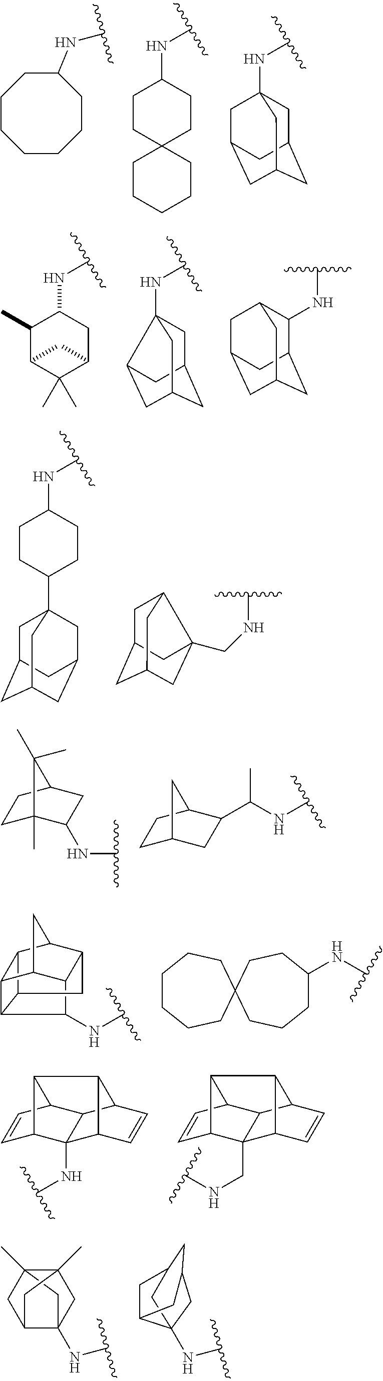

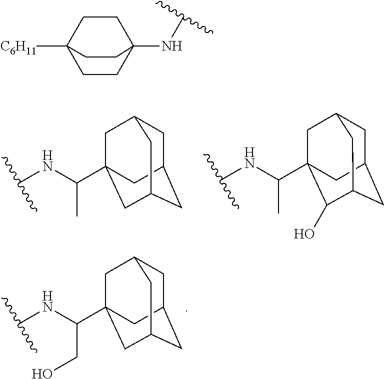

17. The composition of claim 1 or salt thereof, wherein each L¹ is independently G¹-Y²—N(R¹)—Y¹—X¹, and G¹-Y²—N(R¹)— is selected from:

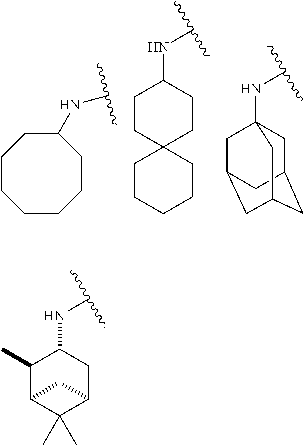

18. The composition of claim 1 or salt thereof, wherein each L¹ is independently G¹-Y²—N(R¹)—Y¹—X¹, and G¹-Y²—N(R¹)— is selected from:

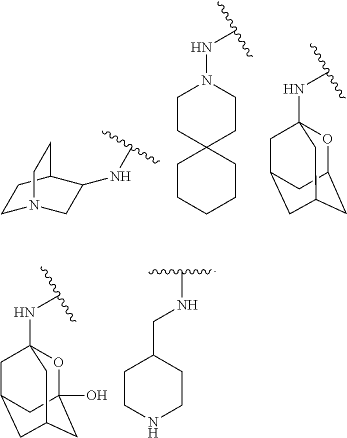

19. The composition of claim 1 or salt thereof, wherein each L¹ is independently G¹-Y²—N(R¹)—Y¹—X¹, and G¹-Y²—N(R¹)— is selected from:

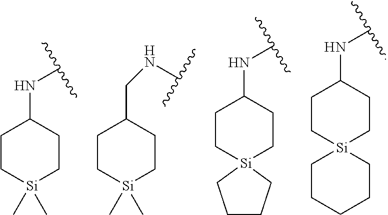

20. The composition of claim 1 or salt thereof, wherein each L¹ is independently G¹-Y²—N(R¹)—Y¹—X¹, and G¹-Y²—N(R¹)— is selected from:

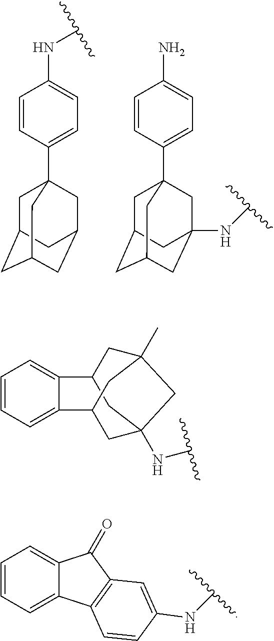

21. The composition of claim 1 or salt thereof, wherein each L¹ is independently G¹-Y²—N(R¹)—Y¹—X¹, and G¹-Y²—N(R¹)— is selected from:

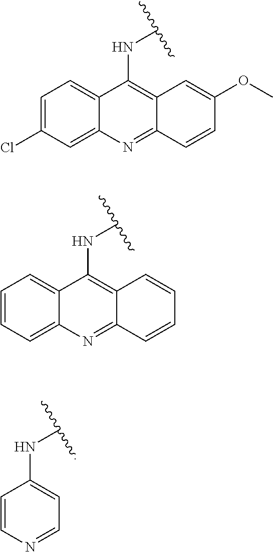

22. The composition of claim 1 or salt thereof, wherein each L¹ is independently G¹-Y²—N(—Y¹—X¹)₂.

23. The composition of claim 1 or salt thereof, wherein each L¹ is independently G¹-Y²—N(—Y¹—X¹)₂, and G¹-Y²—N is selected from:

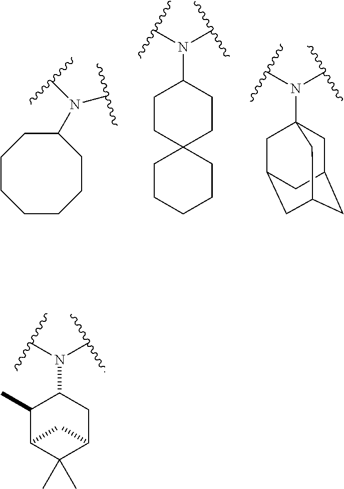

24. (canceled)

25. The composition of claim 1 or salt thereof, wherein L¹ is G²(-Y¹—X¹)ᵣ, and r is 1.

26. The composition of claim 1 or salt thereof, wherein L¹ is G²(-Y¹—X¹)ᵣ, r is 1, and G² is selected from:

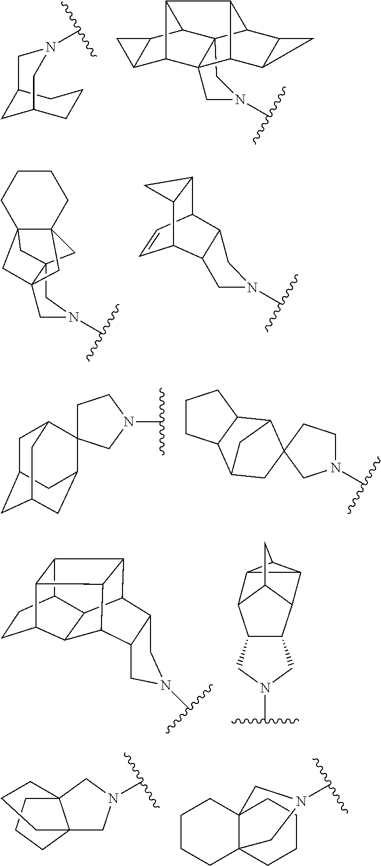

27. The composition of claim 1 or salt thereof, wherein L¹ is G²(-Y¹—X¹)ᵣ, r is 1, and G² is selected from:

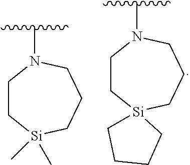

28-30. (canceled)

31. The composition of claim 1 or salt thereof, wherein each L² is selected from the group consisting of water, pyridine, a halide ion, cyanide ion, an acetate ion, phosphate ion, sulfate ion, carbonate ion, bicarbonate ion, nitrate ion, [chemical structure]
